SEQUENCE LISTING (52 sequences)

SEQ ID NO: 1 - protein, 23 aa, Artificial Sequence
  signature peptide of TDP-43 (PN)
IRVTEDENDEPIEIPSEDDGTVL

SEQ ID NO: 2 - protein, 11 aa, Artificial Sequence
  signature peptide of TDP-43 (PC)
SGSNSGAAIGW

SEQ ID NO: 3 - protein, 11 aa, Artificial Sequence
  signature peptide of TDP-43 (PT2)
SNRQLERSGRF

SEQ ID NO: 4 - protein, 23 aa, Artificial Sequence
  isotope labelled amino acid of signature peptide of TDP-43 (PN)
  SITE (2)..(2): isotope labelled amino acid
IRVTEDENDEPIEIPSEDDGTVL

SEQ ID NO: 5 - protein, 11 aa, Artificial Sequence
  isotope labelled amino acid of signature peptide of TDP-43 (PC)
  SITE (6)..(6): isotope labelled amino acid
SGSNSGAAIGW

SEQ ID NO: 6 - protein, 11 aa, Artificial Sequence
  isotope labelled amino acid of signature peptide of TDP-43 (PT2)
  SITE (10)..(10): isotope labelled amino acid
SNRQLERSGRF

SEQ ID NO: 7 - protein, 149 aa, Homo sapiens
SNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 8 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 9 - protein, 13 aa, Artificial Sequence
  signature peptide of TDP-43 (PT1)
KLPNSKQSQDEPL

SEQ ID NO: 10 - protein, 13 aa, Artificial Sequence
  isotope labelled amino acid of signature peptide of TDP-43 (PT1)
  SITE (1)..(1): isotope labelled amino acid
KLPNSKQSQDEPL

SEQ ID NO: 11 - protein, 12 aa, Artificial Sequence
  signature peptide of TDP-43
TSDLIVLGLPWK

SEQ ID NO: 12 - protein, 12 aa, Artificial Sequence
  isotope labelled amino acid of signature peptide of TDP-43
  SITE (12)..(12): isotope labelled amino acid
TSDLIVLGLPWK

SEQ ID NO: 13 - protein, 18 aa, Artificial Sequence
  signature peptide of TDP-43
FGGNPGGFGNQGGFGNSR

SEQ ID NO: 14 - protein, 18 aa, Artificial Sequence
  isotope labelled amino acid of signature peptide of TDP-43
  SITE (18)..(18): isotope labelled amino acid
FGGNPGGFGNQGGFGNSR

SEQ ID NO: 15 - protein, 16 aa, Artificial Sequence
  signature peptide of TDP-43
FGGNPGGFGNQGGFGN

SEQ ID NO: 16 - protein, 16 aa, Artificial Sequence
  isotope labelled amino acid of signature peptide of TDP-43
  SITE (6)..(6): isotope labelled amino acid
FGGNPGGFGNQGGFGN

SEQ ID NO: 17 - protein, 14 aa, Artificial Sequence
  signature peptide of TDP-43
PGGFGNQGGFGNSR

SEQ ID NO: 18 - protein, 14 aa, Artificial Sequence
  isotope labelled amino acid of signature peptide of TDP-43
  SITE (14)..(14): isotope labelled amino acid
PGGFGNQGGFGNSR

SEQ ID NO: 19 - protein, 6 aa, Artificial Sequence
  signature peptide of TDP-43
SSGWGM

SEQ ID NO: 20 - protein, 6 aa, Artificial Sequence
  isotope labelled amino acid of signature peptide of TDP-43
  SITE (3)..(3): isotope labelled amino acid
SSGWGM

SEQ ID NO: 21 - protein, 317 aa, Artificial Sequence
  TDP-43 QconCat Construct
HHHHHHLKSWPESDATVDAFKADGTVPESSGAGLRVAYPSAADIGDYRVIAAPDIITGFRTVQENGGLRIVGGPDIITGFRYPEGGSIVTPYRYPEAGTIVPTYKANSEDVGTEAVFKNSEVGAIIYAAEDATNEKQALSREDAEGRLTSVGSITSGYEYPGSQGSPAVDKERFEVDKIAQNLASSSDTRVGAPATGSGTPGPFTRTSVSSVSASPSRFRGAGAASSTDSLAQEAEAARVDLIRVTEDENDEPIEIPSEDDGTVLSGSNSGAAIGWKLPNSKQSQDEPLSNRQLERSGRFIIKGISVHISNAEPKHN

SEQ ID NO: 22 - protein, 12 aa, Artificial Sequence
  signature peptide of human NFL
AAEDATNEKQAL

SEQ ID NO: 23 - protein, 9 aa, Artificial Sequence
  signature peptide of human NFL
SREDAEGRL

SEQ ID NO: 24 - protein, 10 aa, Artificial Sequence
  signature peptide of human NFL
TSVGSITSGY

SEQ ID NO: 25 - protein, 8 aa, Artificial Sequence
  signature peptide of human NFL
SAPVSSSL

SEQ ID NO: 26 - protein, 10 aa, Artificial Sequence
  signature peptide of human NFL
SQVAAISNDL

SEQ ID NO: 27 - protein, 11 aa, Artificial Sequence
  signature peptide of human NFL
KSIRTQEKAQL

SEQ ID NO: 28 - protein, 10 aa, Artificial Sequence
  signature peptide of human NFL
RQKHSEPSRF

SEQ ID NO: 29 - protein, 16 aa, Artificial Sequence
  signature peptide of human CHIT1
EYPGSQGSPAVDKERF

SEQ ID NO: 30 - protein, 9 aa, Artificial Sequence
  signature peptide of human CHIT1
EVDKIAQNL

SEQ ID NO: 31 - protein, 21 aa, Artificial Sequence
  signature peptide of human CHIT1
ASSSDTRVGAPATGSGTPGPF

SEQ ID NO: 32 - protein, 15 aa, Artificial Sequence
  signature peptide of human CHIT1
KGATKQRIQDQKVPY

SEQ ID NO: 33 - protein, 8 aa, Artificial Sequence
  signature peptide of human CHIT1
LPSGTPEL

SEQ ID NO: 34 - protein, 22 aa, Artificial Sequence
  signature peptide of human CHIT1
EVPKPGQPSEPEHGPSPGQDTF

SEQ ID NO: 35 - protein, 15 aa, Artificial Sequence
  signature peptide of human NFH
TRTSVSSVSASPSRF

SEQ ID NO: 36 - protein, 12 aa, Artificial Sequence
  signature peptide of human NFH
RGAGAASSTDSL

SEQ ID NO: 37 - protein, 11 aa, Artificial Sequence
  signature peptide of human NFH
AQEAEAARVDL

SEQ ID NO: 38 - protein, 19 aa, Artificial Sequence
  signature peptide of human NFH
ARKGGAGGTRSAAGSSSGF

SEQ ID NO: 39 - protein, 17 aa, Artificial Sequence
  signature peptide of human NFH
DDEARQREEAEAAARAL

SEQ ID NO: 40 - protein, 9 aa, Artificial Sequence
  signature peptide of human NFH
EGHAVQSTL

SEQ ID NO: 41 - protein, 11 aa, Artificial Sequence
  signature peptide of human NFH
EDRHQADIASY

SEQ ID NO: 42 - protein, 13 aa, Artificial Sequence
  a retention time standard for mass spectrometry
KSWPESDATVDAF

SEQ ID NO: 43 - protein, 14 aa, Artificial Sequence
  a retention time standard for mass spectrometry
KADGTVPESSGAGL

SEQ ID NO: 44 - protein, 13 aa, Artificial Sequence
  a retention time standard for mass spectrometry
RVAYPSAADIGDY

SEQ ID NO: 45 - protein, 12 aa, Artificial Sequence
  a retention time standard for mass spectrometry
RVIAAPDIITGF

SEQ ID NO: 46 - protein, 9 aa, Artificial Sequence
  a retention time standard for mass spectrometry
RTVQENGGL

SEQ ID NO: 47 - protein, 12 aa, Artificial Sequence
  a retention time standard for mass spectrometry
RIVGGPDIITGF

SEQ ID NO: 48 - protein, 12 aa, Artificial Sequence
  a retention time standard for mass spectrometry
RYPEGGSIVTPY

SEQ ID NO: 49 - protein, 12 aa, Artificial Sequence
  a retention time standard for mass spectrometry
RYPEAGTIVPTY

SEQ ID NO: 50 - protein, 12 aa, Artificial Sequence
  a retention time standard for mass spectrometry
KANSEDVGTEAV

SEQ ID NO: 51 - protein, 11 aa, Artificial Sequence
  a retention time standard for mass spectrometry
FKNSEVGAIIY

SEQ ID NO: 52 - protein, 17 aa, Artificial Sequence
  signature peptide of TDP-43 (PT3)
IIKGISVHISNAEPKHN

(19) United States
(12) Patent Application Publication
(10) Pub. No.: US 2023/0160913 A1
(43) Pub. Date: May 25, 2023

(54) METHOD FOR DIAGNOSING A CONDITION CHARACTERIZED BY TDP-43 PROTEINOPATHY

(71) Applicant: Oxford University Innovation Limited, Oxford (GB)

(72) Inventors: Emily FENEBERY, Buchenberg (DE); Martin TURNER, Oxford (GB); Kevin TALBOT, Oxford (GB)

(21) Appl. No.: 17/916,203

(22) PCT Filed: Apr. 1, 2021
(86) PCT No.: PCT/GB2021/050821

§ 371 Date: Sep. 30, 2022

(30) Foreign Application Priority Data
Apr. 2, 2020 (GB) 2004863.3

(51) Int. Cl. G01N 33/68; C07K 14/47

(52) U.S. Cl. CPC G01N 33/6896; C07K 14/4702; G01N 33/6848; G01N 2800/52

(57) ABSTRACT

The invention relates to the diagnosis of a condition characterised by TDP-43 proteinopathy by mass spectrometry using one or more signature peptides of TDP-43 species.

### FIELD OF THE INVENTION

The invention relates to methods, compositions and kits for diagnosing a condition characterised by TDP-43 proteinopathy.

### BACKGROUND TO THE INVENTION

The presence of ubiquitylated cytoplasmic inclusions of the 43 kDa transactive region DNA-binding protein, TDP-43, is the neuropathological hallmark of the neurodegenerative disorder amyotrophic lateral sclerosis (ALS), and found in 97% of all cases and 50% of cases of frontotemporal dementia (FTD) (1,2). TDP-43 histopathology also defines limbic-predominant age-related TDP-43 encephalopathy (LATE)′ which may coexist with Alzheimer's disease (AD) neuropathological change (3). However, the principal neuroanatomical distribution of microscopic TDP-43 pathology in LATE is distinct from that of classical ALS-TDP.

The neuropathological characteristics of TDP-43 proteinopathy in ALS and FTD are nuclear to cytoplasmic mislocalization, post-translational modifications such as ubiquitination and phosphorylation, aggregation and importantly N-Terminal truncation of TDP-43 resulting in smaller C-terminal TDP-43 fragments (1,4,5). When extracting the insoluble protein fraction from post mortem tissue with TDP-43 proteinopathy, a pathological signature can be detected by immunoblotting, which is characterized by a 45 kDa full-length TDP-43 band, a higher molecular mass smear representing post-translational modifications and lower molecular weight bands at 20-35 kDa which have been identified as C-Terminal TDP-43 fragments (1,6).

Although TDP-43 neuropathology has been well defined using immunohistochemistry (7), biochemical detection of the pathological TDP-43 forms has so far been limited to immunoblotting of the insoluble protein fractions from post mortem tissue and has not yet been reproduced in biofluids (8). Studies using antibodies against TDP-43 and its phosphorylated form to measure pathological TDP-43 by ELISA in cerebrospinal fluid or serum have provided inconsistent findings (9,10,11). Limitations are the significantly lower amounts of pathological TDP-43 present in a complex matrix such as cerebrospinal fluid (CSF) or serum, non-specific binding of commercial antibodies to immunoglobulins, and the limited availability of antibodies that specifically detect pathological, brain-derived TDP-43, including discriminating normal full-length TDP-43 from pathological truncated C-terminal TDP-43 fragments (9,12,13). There is a need for new tests to diagnose, monitor or provide prognosis of a condition characterised by TDP-43 proteinopathy. It is an objective of the invention to meet these needs.

### SUMMARY OF THE INVENTION

The inventors developed a method to detect aberrant TDP-43 species produced under pathological conditions using biochemical techniques, which can be used to diagnose, monitor or provide prognosis of a condition characterised by TDP-43 proteinopathy.

In particular, biochemical fractionated post mortem cortical and spinal cord tissues were used to analyse insoluble proteins (urea fraction) by LC-MS/MS to identify signature peptides of the TDP-43 species (e.g. full-length and low molecular weight TDP-43 fragments). The inventors identified peptide sequences (e.g. the N-terminal peptide (P_{N}; SEQ ID NO: 1), the C-terminal peptide (P_{C}; SEQ ID NO: 2), the N-terminal truncation site peptide (P_{T2}; SEQ ID NO: 3) and the C-terminal truncation site peptide (P_{T3}; SEQ ID NO: 52) that are useful in identifying the enrichment of aberrant TDP-43 species in samples of patients having conditions characterised by TDP-43 proteinopathy. The identified signature peptides were synthesized with a isotope-label isotope tag (heavy) for absolute quantification of corresponding endogenous TDP-43 peptides (light) by parallel reaction monitoring (PRM) in urea fractions from neuropathologically confirmed post mortem tissue of patients having amyotrophic lateral sclerosis (ALS), Parkinson's Disease (PD) and Alzheimer's Disease (AD), and healthy controls (CTL).

In particular, the inventors were able to discriminate ALS from other neurodegenerative diseases, such as AD or PD. The inventors found that the light-heavy ratio of P_{N} was decreased in ALS compared to AD (p=0.001), while the level of P_{C} was increased in ALS compared to PD (p=0.0045). When calculating the ratio of the level of P_{C} to the level of P_{N} to determine the enrichment of C-terminal TDP-43 fragments in ALS urea fractions, a significant increase was observed when ALS was compared to the healthy controls (CTL), AD, PD (p<0.0001). A cut-off value of a ratio over 1.53 discriminated ALS from CTL, at sensitivity of 100% (CI 79.61% to 100%) and 100% specificity (CI 67.56% to 100%), and PD or AD at a sensitivity of 93.3% (CI 70.18% to 99.66%) and specificity of 100% (CI 67.56% to 100%).

The ratio of the level of a further signature peptide, P_{T1} (SEQ ID NO: 9), to the level of P_{N} in ALS samples is also useful in diagnosing TDP-43 proteinopathy. This ratio was significantly increased in ALS compared to CTL, AD, PD (p=0.001, p=0.04 and p<0.0001, respectively).

Furthermore, the inventors identified the accumulation of a C-terminal TDP-43 fragment (SEQ ID NO: 7) with a new truncation site at the N-terminal, and its signature peptide (e.g. P_{T2}) can be used to discriminate ALS from other neurodegenerative diseases, such as AD or PD. In particular, the inventors found that the light to heavy ratio of P_{T2} was increased in ALS compared to PD and CTL (p<0.002 and p=0.01, respectively), but decreased compared to AD (0.01). When calculating the ratio of the level of P_{T2} to the level of P_{N}, a significant increase was observed when ALS was compared to the healthy controls (CTL), PD (p=0.007 and p=0.004, respectively). A cut-off value of a ratio over 0.004 discriminated ALS from PD with a sensitivity of 100% (CI 70% to 100%) and 100% specificity (CI 18% to 100%), and CTL with a sensitivity of 88.89 (CI 56.50% to 99.43%) and 67% specificity (CI 11.85% to 98.29%). A cut-off value below 0.016 discriminated ALS from AD with a sensitivity of 77.78 (CI 45.26% to 96.05%) and specificity of 100% (CI 60.97% to 100.0%).

Thus, the invention provides a method for analysing a sample from a subject, comprising detecting a signature peptide of a TDP-43 species in the sample, wherein the signature peptide is a fragment of TDP-43 that is 6 to 25 amino acids in length comprising an amino acid sequence having at least 90% sequence identity to the amino acid sequence of any of SEQ ID NOs: 1-3 and 52, and wherein the level of the signature peptide provides a diagnostic indicator of a subject having a condition characterised by TDP-43 proteinopathy.

The invention also provides a method for diagnosing if a subject has a condition characterised by TDP-43 proteinopathy, comprising analysing a sample from the subject according to the method of the invention.

The invention also provides a method of discriminating a condition characterised by TDP-43 proteinopathy from a condition characterised by a proteinopathy other than TDP-43 proteinopathy, comprising analysing a sample from the subject according to the method of the invention.

The invention also provides a method of discriminating amyotrophic lateral sclerosis from other forms of TDP-43 proteinopathy, e.g. Limbic-predominant age-related TDP-43 encephalopathy, comprising analysing a sample from the subject according to the method of the invention.

The inventors can discriminate AD from ALS with the identified peptide sequences (e.g. the N-terminal peptide (P_{N}; SEQ ID NO: 1), the C-terminal peptide (P_{C}; SEQ ID NO: 2), the N-terminal truncation site peptide (P_{T2}; SEQ ID NO: 3) that are useful in identifying the enrichment of aberrant C-terminal fragment TDP-43 species in samples of patients having conditions characterised by different TDP-43 proteinopathy. The inventors were able to discriminate ALS from AD. The inventors found that the light-heavy ratio of P_{N} was decreased in ALS compared to AD (p=0.001). When calculating the ratio of the level of P_{C} to the level of P_{N} to determine the enrichment of C-terminal TDP-43 fragments in ALS urea fractions, a significant increase was observed when ALS was compared to AD (p<0.0001). A cut-off value of a ratio over 1.53 discriminated ALS from CTL, at sensitivity of 100% (CI 79.61% to 100%) and 100% specificity (CI 67.56% to 100%), and PD or AD at a sensitivity of 93.3% (CI 70.18% to 99.66%) and specificity of 100% (CI 67.56% to 100%). In addition, The inventors also found that the light to heavy ratio of P_{T2} was increased in ALS compared to PD and CTL (p<0.002 and p=0.01, respectively), but decreased compared to AD (0.01). A cut-off value below 0.016 discriminated ALS from AD with a sensitivity of 77.78 (CI 45.26% to 96.05%) and specificity of 100% (CI 60.97% to 100.0%).

The invention also provides a method of monitoring the efficacy of a therapy for a condition characterised by TDP-43 proteinopathy being administered to a subject, comprising analysing a sample from the subject according to the method of the invention, wherein the level of the signature peptide is determined at two or more different points in time, with changing levels of the TDP-43 species over time indicating whether the disease is getting better or worse.

The invention also provides a method of determining the prognosis of a condition characterised by TDP-43 proteinopathy in a subject, comprising analysing a sample from the subject according to the method of the invention, wherein the level of the TDP-43 species is determined at two or more different points in time, with changing levels of the signature peptide over time indicating whether the disease is getting better or worse.

The invention also provides a method of identifying a subject susceptible to a therapy for Alzheimer's disease, e.g. immunisation against Tau or Amyloid, or antisense treatment targeted to tau, comprising diagnosing if a subject has a condition characterised by TDP-43 proteinopathy according to the method of the invention, wherein the subject having a condition characterised by TDP-43 proteinopathy is not susceptible to the therapy.

The invention also provides a method of treating a condition characterised by TDP-43 proteinopathy, comprising administering a nucleic acid molecule targeted to a gene causing TDP-43 proteinopathy, a neuroprotective agent, an anti-inflammatory agent, an agent that regulates inflammation or immune system, an agent that protect cells from accumulation of misfolded protein or excitatory stimuli or high-caloric treatment to the subject, wherein the method further comprises diagnosing if a subject has a condition characterised by TDP-43 proteinopathy according to the method of the invention, monitoring the efficacy of the therapy according to the method of the invention, and/or determining the prognosis of the disease according to the method of the invention.

The invention also provides a nucleic acid molecule targeted to a gene causing TDP-43 proteinopathy, an anti-inflammatory agent, an agent that regulates inflammation or immune system, or an agent that protect cells from accumulation of misfolded protein or excitatory stimuli for use in a method of treating a condition characterised by TDP-43 proteinopathy, comprising administering said molecule or agent to the subject, wherein the method further comprises diagnosing if a subject has a condition characterised by TDP-43 proteinopathy according to the method of the invention, monitoring the efficacy of the therapy according to the method of the invention, and/or determining the prognosis of the disease according to the method of the invention.

The invention also provides a signature peptide of TDP-43 which is a fragment of TDP-43 that is 6 to 25 amino acids in length comprising an amino acid sequence having at least 90% sequence identity to the amino acid sequence of any of SEQ ID NOs: 1-3 and

The invention also provides an internal standard for detecting a signature peptide of TDP-43, wherein the internal standard is an isotope-labelled signal peptide of the invention.

The invention also provides an internal standard precursor, comprising the internal standard of the invention.

The invention also provides a fragment of TDP-43 which is 100 to 180 amino acids in length comprising an amino acid sequence having at least 90% sequence identity to the amino acid sequence of SEQ ID NO: 7.

The invention also provides a polynucleotide encoding the peptide of the invention.

The invention also provides an antibody capable of binding specifically to the peptide of the invention.

The invention also provides the use of the signature peptide of the invention as a biomarker for a TDP-43 species.

The invention also provides the use of the internal standard, the internal standard precursor or the antibody of the invention to diagnose or provide prognosis for a condition characterised by TDP-43 proteinopathy.

### BRIEF DESCRIPTION OF THE FIGURES

FIG. 1 shows the neuropathology of the study cohort. (A) ALS-TDP with granular-, skein- and neuritic pTDP-43 aggregates in the motor cortex (layer three) and nucleus hypoglossus (inset). (B) PD with alpha-synuclein-positive Lewy bodies, granular deposits and Lewy neurites in the substantia nigra (inset: pigmented dopaminergic neuron with Lewy body H&E). (C)-(D) classical AD with a neuritic beta-amyloid-positive plaque (C) and neurofibrillary tangles in the granule cells of the hippocampus (D) (serial sections). Abbreviations: ALS amyotrophic lateral sclerosis, AD Alzheimer's disease, PD Parkinson's disease, pTDP-43 phosphorylated TDP-43.

FIG. 2 shows the quantification of C- and N-Terminal TDP-43 peptides by parallel reaction monitoring discriminates ALS from other neurodegenerative diseases. (A) The chymotryptic N-Terminal peptide ratio is significantly decreased in ALS compared to AD (**p=0.001), but not in ALS compared to CTL and PD (p=0.13 and p=0.051). (B) The chymotryptic C-Terminal peptide ratio is significantly increased in ALS compared to PD (**p=0.0045), but not in ALS compared to AD and CTL (p=0.7, p=0.29). (C) The C- to N-Terminal peptide ratio is significantly increased in ALS compared to AD, PD and CTL (****p<0.0001 respectively). (D) ROC curves of N-Terminal and C-Terminal peptide ratios and the calculated C- to N-terminal peptide ratio for discrimination between ALS and CTL, PD or AD. For comparison, the AUCs are shown. Abbreviations: ALS amyotrophic lateral sclerosis, ALS Plus ALS with more sever TDP-43 pathology, AD Alzheimer's disease, PD Parkinson's disease, CTL healthy control, ROC receiver operating characteristics, AUC area under the curve. Significance of ALS versus all groups was determined by one-way ANOVA with Dunnett's multiple comparison test.

FIG. 3 shows the quantification of Truncation site specific TDP-43 peptides by parallel reaction monitoring identifies pathological TDP-43 processing in ALS and AD. (A) The Truncation 1 peptide ratio is significantly increased in ALS compared to PD and CTL (****p<0.0001, ***p=0.0008), but not in ALS compared to AD (p=0.94). The Truncation 1 to N-Terminal peptide ratio is significantly increased in ALS compared to AD, PD and CTL (*p=0.04, ****p<0.0001 and p=0.001). (B) ROC curves of the Truncation 1 peptide ratio and the calculated Truncation 1 to N-terminal peptide ratio for discrimination between ALS and CTL, PD or AD. For comparison, the AUCs are shown. (C) The Truncation 2 peptide ratio is significantly increased in ALS compared to PD and CTL (**p<0.002, *p=0.01), but decreased compared to AD (*p=0.01). The Truncation 2 to N-Terminal peptide ratio is significantly increased in ALS compared to PD and CTL (**p=0.004, **p=0.007), but not AD (p=0.79). (D) ROC curves of the Truncation 2 peptide ratio and the calculated Truncation 2 to N-terminal peptide ratio for discrimination between ALS and CTL, PD or AD. For comparison, the AUCs are shown. Abbreviations: ALS amyotrophic lateral sclerosis, AD Alzheimer's disease, PD Parkinson's disease, CTL healthy control, ROC receiver operating characteristics, AUC area under the curve. Significance of ALS versus all groups was determined by one-way ANOVA with Dunnett's multiple comparison test.

FIG. 4 shows that the C-Terminal fragments at 25 kDa are increased in insoluble urea fractions extracted from ALS cortex tissue. Shown are representative immunoblots with an anti-C-Terminal TDP-43 antibody of the soluble (TX) protein fractions and the insoluble (urea) protein fractions extracted from cortex tissue of the CTL, ALS, PD and AD cohort used for parallel reaction monitoring. (A) Quantification of immunoreactive bands at 43 kD, 35 kDa and 25 kDa shows equal ratios of CTF-35 to FL TDP-43 and CTF-25 to FL TDP-43 between diagnostic groups. (B) Quantification of immunoreactive bands at 43 kD, 35 kDa and 25 kDa shows equal ratios of CTF-35 to full-length TDP-43 between diagnostic groups, but increased CTF-25 to full-length TDP-43 in ALS. Abbreviations: FL full-length, CTF C-Terminal fragments.

FIG. 5 shows coexisting LATE neuropathological change in AD brain. pTDP-43 aggregates in (A) amygdala, (B) entorhinal cortex and (C) hippocampal granule cells. No pTDP43 aggregates are visible in primary motor cortex (D) and hypoglossal nucleus (E, inset). Scale bar 20 μm.

FIG. 6 shows the C- to N-Terminal TDP-43 peptide ratio is not increased in ALS spinal cord urea fractions. (A) In contrast to cortex urea fractions the chymotryptic N-Terminal peptide ratio is significantly increased in ALS compared to CTL (***p=0.0001), but not in ALS compared to PD and AD (p=0.14 and p=0.88). (B) The chymotryptic C-Terminal peptide ratio is unaltered in ALS compared to all other diagnostic groups (CTL p=0.3, PD and AD p=0.9). (C) The C- to N-Terminal peptide ratio is not increased in ALS compared to CTL (p=0.1) PD or AD (both p=0.9).

FIG. 7 shows a schematic drawing of the full length TDP-43, indicating the locations of the peptides described herein.

### BRIEF DESCRIPTION OF THE SEQUENCE LISTING

SEQ ID NO: 1 is the amino acid sequence of a signature peptide of TDP-43 (P_{N}).
SEQ ID NO: 2 is the amino acid sequence of a signature peptide of TDP-43 (P_{C}).
SEQ ID NO: 3 is the amino acid sequence of a signature peptide of a truncated TDP-43 (P_{T2}).
SEQ ID NO: 4 is an isotope-labelled amino acid sequence of SEQ ID NO: 1.
SEQ ID NO: 5 is an isotope-labelled amino acid sequence of SEQ ID NO: 2.
SEQ ID NO: 6 is an isotope-labelled amino acid sequence of SEQ ID NO: 3.
SEQ ID NO: 7 is the amino acid sequence of a TDP-43 fragment.
SEQ ID NO: 8 is the amino acid sequence of full length TDP-43.
SEQ ID NO: 9 is the amino acid sequence of a signature peptide of TDP-43 (P_{T1}).
SEQ ID NO: 10 is an isotope-labelled amino acid sequence of SEQ ID NO: 9.
SEQ ID NO: 11 is the amino acid sequence of a signature peptide of TDP-43.
SEQ ID NO: 12 is an isotope-labelled amino acid sequence of SEQ ID NO: 11.
SEQ ID NO: 13 is the amino acid sequence of a signature peptide of TDP-43.
SEQ ID NO: 14 is an isotope-labelled amino acid sequence of SEQ ID NO: 13.
SEQ ID NO: 15 is the amino acid sequence of a signature peptide of TDP-43.
SEQ ID NO: 16 is an isotope-labelled amino acid sequence of SEQ ID NO: 15.
SEQ ID NO: 17 is the amino acid sequence of a signature peptide of TDP-43.
SEQ ID NO: 18 is an isotope-labelled amino acid sequence of SEQ ID NO: 17.
SEQ ID NO: 19 is the amino acid sequence of a signature peptide of TDP-43.
SEQ ID NO: 20 is an isotope-labelled amino acid sequence of SEQ ID NO: 19.
SEQ ID NO: 21 is the amino acid sequence of an internal standard precursor.
SEQ ID NOs: 22-28 are signature peptides of human neurofilament light polypeptide (NFL).
SEQ ID NOs: 29-34 are signature peptides of human chitotriosidase-1 (CHIT1).
SEQ ID NOs: 35-41 are signature peptides of human neurofilament heavy polypeptide (NFH).
SEQ ID NOs: 42-51 are retention time standards for mass spectrometry.
SEQ ID NO: 52 is a signature peptide of TDP-43 (P_{T3}).

### DETAILED DESCRIPTION OF THE INVENTION

### TDP-43 Species and Signature Peptides

The invention involves detecting a signal peptide of a TDP-43 species in a sample. TDP-43 species comprise the full length human TDP-43 (SEQ ID NO: 8), which has 414 amino acid residues (43 kDa), higher molecular mass TDP-43 species resulting from post-translational modifications such as ubiquitination and phosphorylation, and/or lower molecular mass TDP-43 species which may be C-terminal TDP-43 fragments resulting from N-Terminal truncation of TDP-43, e.g. SEQ ID NO: 7. A TDP-43 species may be an aberrant TDP-43 species produced under pathological conditions. Hence, a TDP-43 species may be specific to a condition characterised by TDP-43 proteinopathy, such as the TDP-43 fragment as set forth in SEQ ID NO:7. Furthermore, point mutations have been found in TDP-43 in disease patients, e.g. see reference 14, and so a TDP-43 species may be a TDP-43 protein containing point mutations.

A TDP-43 species can be identified by one or more signature peptides. A TDP-43 signature peptide of the invention may be selected based on several criteria. For example, a signature peptide is selected based on its specificity, including the correct identification in chymotrypsin/trypsin digested samples, small chromatographic shifts and zero divergence from the corresponding heavy-labelled peptide, which made them the strongest candidates for quantification.

A signature peptide of the invention can be useful as a biomarker of an aberrant TDP-43 species produced under pathological conditions. Hence, a signature peptide of the invention may be useful in aiding diagnosis or prognosis of a condition characterised by TDP-43 proteinopathy.

A signature peptide of the invention is a fragment of TDP-43 that is 6 to 25 amino acids in length comprising an amino acid sequence having at least 90% sequence identity to the amino acid sequence of any of SEQ ID NOs: 1-3 and 52.

The signature peptide is a fragment of TDP-43. It is at least 6 amino acids in length. It may be up to 11, up to 17 or up to 23 amino acids in length. For example, it may be 6, 7, 8, 9, 10, 11, 12, 13, 14, 15, 16, 17, 18, 19, 20, 21, 22, 23, 24 or 25 amino acids in length.

The signature peptide may comprise an amino acid sequence having at least 90%, 95%, at least 99% or 100% sequence identity to the amino acid sequence of any of SEQ ID NOs: 1-3 and 52.

For example, the signature peptide may differ from any of SEQ ID NOs: 1-3 and 52 by one amino acid or two amino acids.

The signature peptide may comprise at least 4, at least 5, at least 6, at least 7, at least 8, at least 9, at least 10, at least 11, at least 12, at least 13, at least 14, at least 15, at least 16, at least 17, at least 18, at least 19, at least 20, at least 21 or at least 22 contiguous amino acids of SEQ ID NO: 1. The signature peptide may comprise or consist of the amino acid sequence of SEQ ID NO: 1.

The signature peptide may comprise at least 4, at least 5, at least 6, at least 7, at least 8, at least 9 or at least 10 contiguous amino acids of SEQ ID NO: 2. The signature peptide may comprise or consist of the amino acid sequence of SEQ ID NO: 2.

The signature peptide may comprise at least 4, at least 5, at least 6, at least 7, at least 8, at least 9 or at least 10 contiguous amino acids of SEQ ID NO: 3. The signature peptide may comprise or consist of the amino acid sequence of SEQ ID NO: 3.

The signature peptide may comprise at least 4, at least 5, at least 6, at least 7, at least 8, at least 9, at least 10, at least 11, at least 12, at least 13, at least 14, at least 15 or at least 16 contiguous amino acids of SEQ ID NO: 52. The signature peptide may comprise or consist of the amino acid sequence of SEQ ID NO: 52.

The invention involves detecting a signature peptide of the invention. In particular, the invention involves detecting a signature peptide which is a fragment of TDP-43 that is 6 to 25 amino acids in length comprising the amino acid sequence of: P_{N} (SEQ ID NO: 1), P_{C} (SEQ ID NO: 2), P_{T2} (SEQ ID NO: 3) and/or P_{T3} (SEQ ID NO: 52). The invention may additionally involve detecting other signature peptides of TDP-43, each of which is a fragment of TDP-43 that is 6 to 25 amino acids in length comprising the amino acid sequence of SEQ ID NOs: 9, 11, 13, 15, 17 and/or 19.

The locations of the signature peptides within TDP-43 are shown in FIG. 7.

The invention may involve detecting the signature peptide in isolated form, or as part of the TDP-43 protein and/or fragments thereof.

The level of a signature peptide can be correlated with the level of the TDP-43 species comprising the signature peptide. Thus, the invention also involves detecting a TDP-43 species that comprises a signature peptide described herein. In particular, a method of the invention involves detecting a TDP-43 species that comprises any of P_{N} (SEQ ID NO: 1), P_{C} (SEQ ID NO: 2), P_{T2} (SEQ ID NO: 3) and/or P_{T3} (SEQ ID NO: 52). The invention may additionally involve detecting a TDP-43 species that comprises the signature peptide of any of SEQ ID NOs: 9, 11, 13, 15, 17 and/or 19.

To improve diagnostic power, the invention may involve detecting two or more (e.g. ≥2, ≥3, ≥4 or ≥5) signature peptides of TDP-43. For example, the invention may involve the detection of the signature peptide comprising P_{C} (SEQ ID NO: 2) and the signature peptide comprising P_{N} (SEQ ID NO: 1). The invention may involve the detection of the signature peptide comprising P_{N} (SEQ ID NO: 1) and the signature peptide comprising P_{T2} (SEQ ID NO: 3). The invention may involve the detection of the signature peptide comprising P_{N} (SEQ ID NO: 1) and the signature peptide comprising P_{T1} (SEQ ID NO: 9). The invention may involve the detection of the signature peptide comprising P_{C} (SEQ ID NO: 2), the signature peptide comprising P_{N} (SEQ ID NO: 1), the signature peptide comprising P_{T2} (SEQ ID NO: 3) and the signature peptide comprising P_{T1} (SEQ ID NO: 9). The invention may involve the detection of the signature peptide comprising P_{C} (SEQ ID NO: 2), the signature peptide comprising P_{N} (SEQ ID NO: 1), the signature peptide comprising P_{T2} (SEQ ID NO: 3), the signature peptide comprising P_{T1} (SEQ ID NO: 9) and the signature peptide comprising P_{T3} (SEQ ID NO: 52).

Similarly, the invention may involve the detection of a TDP-43 species comprising P_{C} (SEQ ID NO: 2) and a TDP-43 species comprising P_{N} (SEQ ID NO: 1). The invention may involve the detection of a TDP-43 species comprising P_{N} (SEQ ID NO: 1) and a TDP-43 species comprising P_{T2} (SEQ ID NO: 3). The invention may involve the detection of a TDP-43 species comprising P_{N} (SEQ ID NO: 1) and a TDP-43 species comprising P_{T1} (SEQ ID NO: 9). The invention may involve the detection of a TDP-43 species comprising P_{C} (SEQ ID NO: 2), a TDP-43 species comprising P_{N} (SEQ ID NO: 1), a TDP-43 species comprising P_{T2} (SEQ ID NO: 3) and a TDP-43 species comprising P_{T1} (SEQ ID NO: 9). The invention may involve the detection of a TDP-43 species comprising P_{C} (SEQ ID NO: 2), a TDP-43 species comprising P_{N} (SEQ ID NO: 1), a TDP-43 species comprising P_{T2} (SEQ ID NO: 3), a TDP-43 species comprising P_{T1} (SEQ ID NO: 9) and a TDP-43 species comprising P_{T3} (SEQ ID NO: 52).

The invention also provides a fragment of TDP-43 which is 100 to 180 amino acids in length comprising an amino acid sequence having at least 90% sequence identity to the amino acid sequence of SEQ ID NO: 7. The TDP-43 fragment is at least 100 amino acids in length. It may be up to 130, 140 or 150 amino acids in length. The TDP-43 fragment may comprise an amino acid sequence having at least 90%, 95%, at least 99% or 100% sequence identity to the amino acid sequence of SEQ ID NO: 7. For example, the TDP-43 fragment may differ from SEQ ID NO: 7 by 1, 2, 3 or 4 amino acids. The TDP-43 fragment may consist of SEQ ID NO: 7.

### The Sample

The invention involves analysing a sample from a subject. The sample may be a (homogenised) tissue or a body fluid.

In some embodiments, a method of the invention involves an initial step of obtaining the sample from the subject. In other embodiments, however, the sample is obtained separately from and prior to performing a method of the invention. After a sample has been obtained then methods of the invention could be performed in vitro.

Suitable tissues may be tissues from the cortex or spinal cord. A tissue sample can be preserved with a fixative (e.g. formalin) before it is analysed. A preserved sample can also be embedded (e.g. formalin-fixed, paraffin-embedded (FFPE) samples). Alternatively, a fresh tissue sample can be used, and this sample is fresh frozen, without fixatives.

Suitable body fluids may be blood or cerebrospinal fluid (CSF). In certain embodiments, the sample is CSF. The sample may be treated between being taken from a subject and being analysed. For example, a blood sample may be treated by adding anti-coagulants (e.g. EDTA), followed by removing cells and cellular debris, leaving plasma for analysis. Alternatively, a blood sample may be allowed to coagulate, followed by removing cells and various clotting factors, leaving serum for analysis.

The invention may further comprise isolating proteins from the sample. Protein isolation procedures are well known in the art. For example, tissues can be homogenised in the presence of appropriate cold buffers using steel beads (e.g. TissueLyser system, Qiagen), in a blender, polytron or by ultrasonication.

The invention may further comprise separating insoluble fraction from total protein isolated from the sample. The TDP-43 species are typically present in the insoluble protein fraction. The insoluble proteins may be resuspended in a denaturing buffer, such as 7M urea.

The invention may further comprise isolating extracellular vesicles from the fluid sample, e.g. CSF. Extracellular vesicles are double-membrane vesicles (40-120 nm) released by most cell types. The signature peptides are determined from the extracellular vesicles isolated from the fluid sample, e.g. CSF. Methods of isolating extracellular vesicles are well known in the art 15.

The sample may be further treated with a protease, such as trypsin or chymotrypsin, prior to detection of the signature peptides, e.g. by mass spectrometry. In certain embodiments, the sample is treated with chymotrypsin. The treatment may be carried out in gel or in solution. The sample may then be further treated, such as concentrated, desalted, and detergents removed, e.g. by using a solid support, prior to detection of the signature peptides, e.g. by mass spectrometry.

In certain aspects of the invention, the subject has one or more signs or symptoms of a condition characterised by TDP-43 proteinopathy and who has not been diagnosed with the condition. The invention may further comprise a step of identifying a subject having one or more signs or symptoms of a condition characterised by TDP-43 proteinopathy and who has not been diagnosed with the condition.

A condition characterised by TDP-43 proteinopathy encompasses several conditions, such as amyotrophic lateral sclerosis, frontotemporal dementia or Limbic-predominant age-related TDP-43 encephalopathy. Signs and symptoms of a condition characterised by TDP-43 proteinopathy are well described in the art. For example, the signs and symptoms of ALS may comprise fasciculations (muscle twitches) in the arm, leg, shoulder, or tongue; muscle cramps, tight and stiff muscles (spasticity); muscle weakness affecting an arm, a leg, neck or diaphragm; slurred and nasal speech; and/or difficulty chewing or swallowing (e.g. see reference 16). For example, the signs and symptoms, e.g. of frontotemporal dementia, include behavioural and personality changes, typically before the age of 60 years (e.g. see reference 17).

For subjects already displaying some signs and symptoms of a condition characterised by TDP-43 proteinopathy, the invention may be used to confirm or resolve another diagnosis. For example, the subject may be suspected to have other forms of neurodegenerative condition or other conditions that have similar signs and symptoms as TDP-43 proteinopathy, i.e. mimic disorders. Such mimic disorders may be multifocal motor neuropathy, myeloradiculopathy (cervical), absolute stenosis of the spine, (bilateral) neuralgic amyotrophy of the brachial plexus, polyneuropathy with amyloidosis, polyneuropathy with monoclonal gammopathy, inflammatory demyelinating polyneuropathy, facioscapulohumeral muscular dystrophy, or juvenile parkinsonism.

The subject may have already begun treatment. For example, the subject may have begun a therapy for a condition characterised by TDP-43 proteinopathy. In this case, the invention may be useful in monitoring the efficacy of the therapy. Suitable therapies for a condition characterised by TDP-43 proteinopathy are known in the art and are described further below.

The subject may have begun a therapy for Alzheimer's disease, e.g. immunisation studies against Tau or Amyloid, or antisense treatment targeted to tau. In this case, the invention may be useful in identifying the susceptibility of the subject to the therapy because a subject having TDP-43 proteinopathy may not be susceptible to said therapy or may be subject to an adverse response to said therapy.

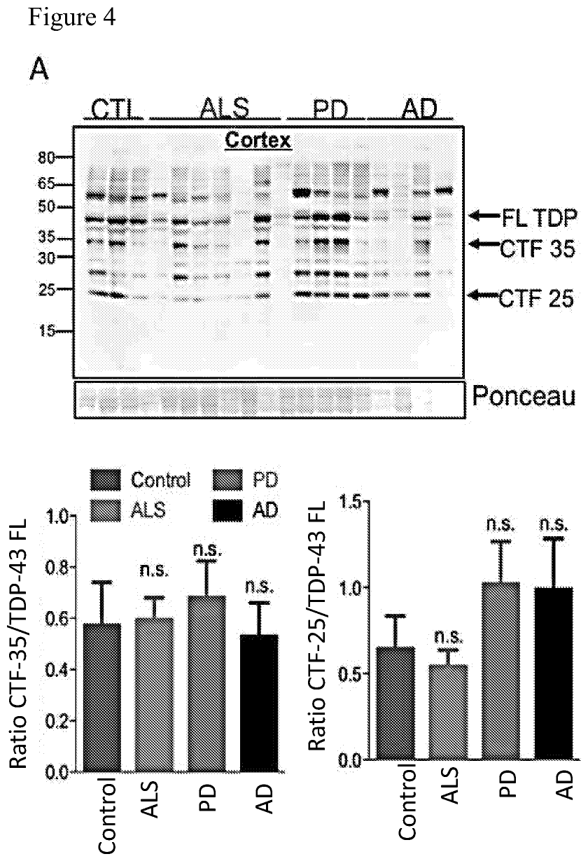

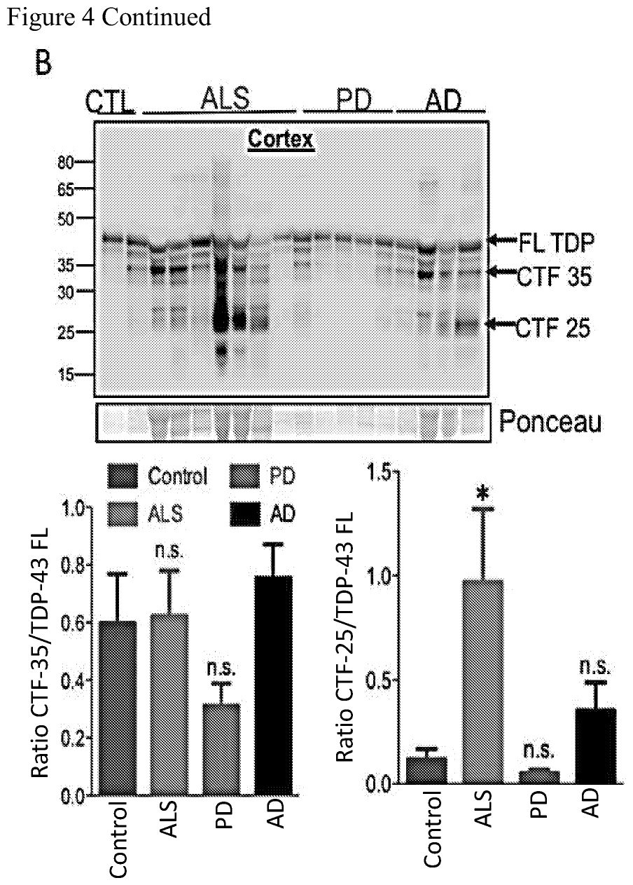

The subject may already be known to be predisposed to the development of a condition characterised by TDP-43 proteinopathy e.g. due to family or genetic links. For example, the subject may contain mutations in the following genes: neurofilament heavy polypeptide (NFH), Alsin, Senataxin (SETX), Spatacsin (SPG11), Vesicle associated membrane protein B (VAPB), angiogenin (ANG), DNA-binding protein (TARDBP), phosophoinositide-5-phosphatease (FIG. 4), optineurin (OPTN), valosin containing protein (VCP), ubiquilin 2 (UBQLN2), sigmarl, C90RF72, dynactin (DCTN1), Sequestosome 1 (SQSTM1), serine/threonine-protein kinase 1 gene (TBK1), Kinesin Family Member 5A (KIF5A), Profilin (PFN1), Tubulin Alpha 4a (TUBA4A), heterogenous Ribonucleoprotein (hnRNP1A) and/or progranulin (GRN). In other embodiments, the subject may have no such predisposition, and may develop the disease as a result of environmental factors e.g. as a result of exposure to particular chemicals (such as toxins or pharmaceuticals), as a result of diet, as a result of infection, etc.

The subject may be predisposed to the development of a condition characterised by TDP-43 proteinopathy, but is yet asymptomatic.

The subject will typically be a human being. In some embodiments, however, the invention is useful in transgenic non-human organisms expressing human TDP-43, e.g. mouse, rat, rabbit, guinea pig, cat, dog, horse, pig, cow, or non-human primate (monkeys or apes, such as macaques or chimpanzees).

The sample may be human induced pluripotent stem-cells (hiPSC) derived from patients and other human cellular models.

Detection

A signature peptide may be detected using any suitable technique that enables specific quantification of the signature peptide in a complex mixture.

For example, a suitable technique may be mass spectrometry. Mass spectrometry is particularly useful in determining the level of a signature peptide, and hence the level of the TDP-43 species comprising a signature peptide. Tandem mass spectrometry, also known as MS/MS, is particularly useful. Liquid chromatography electrospray ionisation tandem mass spectrometry (LC-ESI-MSMS) is a common approach for analysis of complex peptide mixtures.

To enhance the mass resolving and mass determining capabilities of mass spectrometry the mass spectrometry may be combined with separation techniques, such as chromatography (e.g. gas chromatography or liquid chromatography), capillary electrophoresis or ion mobility spectrometry). For example, the tandem mass spectrometry may be combined with separation of the peptides by liquid chromatography, such as high performance liquid chromatography (HPLC) or ultra-high performance liquid chromatography (UHPLC).

For detection by mass spectrometry, the methods of the invention may comprise treating the sample with a specific protease, such as chymotrypsin, in order to generate specific monitorable peptides from TDP43 species. The methods of the invention may further comprise introducing the resultant peptide mixture into the mass spectrometer over time by elution from a low pH reverse-phase column with a specific gradient.

The methods of the invention may further comprise configuration of the mass spectrometer instrument to perform specific mass isolation, fragment ion generation and detection of said fragment ions at defined times (specific to peptide elution from the liquid chromatography gradient). The mass isolation, fragment ion generation and detection actions each involves specific configuration parameters to allow detection of the fragment ion signals diagnostic for the target TDP43 peptides at workable Signal-to-Noise.

A signature peptide may be detected by an antibody-based approach, such as an antibody capable of binding specifically to a peptide of TDP-43 described herein, e.g. a peptide comprising SEQ ID NOs: 1-3, 7 and 52. Hence, the antibody may detect a TDP-43 species comprising the signature peptide.

### Internal Standard

A method of the invention may involve adding an internal standard to the sample prior to analysis, such as by mass spectrometry. The internal standard may serve as an internal reference standard or a calibrant for the analysis. It may be used to determine the absolute amount of the signature peptide in a complex mixture. For example, if the concentration of the internal standard in the sample is known, the level of a biomarker detectable by the method of the present invention (e.g. a signature peptide of a TDP-43 species) can be determined, for example, by determining the ratio of the peak area of the signal peptide and the peak area of the internal standard in a mass spectrum.

Any suitable internal standard can be used in the methods of the present invention. The internal standard is typically a modified, synthetic peptide that has 100% amino acid identity to an endogenous TDP-43 signature peptide, but that is modified to differ only by mass, e.g. by isotope-labelling, with no other difference in physiochemical characteristics. Thus, the internal standard and the corresponding TDP-43 signature peptide have the same chromatographic and ionisation behaviours. The internal peptide can be independently detected, e.g. by mass spectrometry, based on the mass difference compared to the corresponding TDP-43 signature peptide. The internal peptide may be referred to herein as a heavy peptide or a heavy isotope-labelled peptide.

The heavy isotope-labelled peptide may comprise an atom of an amino acid that has been substituted with a stable isotope. For example, any hydrogen, carbon, nitrogen, oxygen or sulphur atom may be replaced with isotopically stable isotope: 2H, 13C, 15 N, 170 and/or ³⁴S. The isotope-labelled peptide may comprise one or more stable isotopes, at least two stable isotopes, or at least three stable isotopes. For example, the heavy isotope-labelled peptide may comprise one or more ¹³C instead of ¹²C, and/or one or more ¹⁵N instead of ¹⁴N. The isotope may be incorporated into one or more atoms in one or more amino acids, such as lysine, arginine and/or glycine.

Methods to generate a heavy isotope-labelled peptide are known in the art. For example, the heavy isotope-labelled peptide may be generated synthetically, e.g. using Fmoc solid-phase peptide technology (18). Alternatively, the heavy isotope-labelled peptide may be generated by QconCAT strategy (32). In brief, the QconCAT strategy involves culturing cells that express the internal standard precursor, where all possible sources of nitrogen for the cell culture in which the protein is synthesised are nearly 100% Nitrogen-15. This way, all the protein synthesised, including the internal standard precursor, are labelled with ¹⁵N.

One or more internal standards can be added to the sample prior to the detection of the signature peptides, such as by mass spectrometry.

Thus, the invention provides an internal standard for detecting a signature peptide of TDP-43, wherein the internal standard is an isotope-labelled signal peptide of the invention. The internal standard may consist of any one of SEQ ID NOs: 4-6.

The invention also provides a method of preparing an internal standard of the invention, comprising labelling an atom of an amino acid of a peptide of the invention with a stable isotope.

The invention also provides an internal standard in a precursor form. For example, the internal standard precursor may comprise multiple internal standards, and each internal standard may be separated, e.g. by enzyme digest, before detection.

For example, the internal standard precursor may comprise: an internal standard for detecting a signature peptide of TDP43 and an internal standard for detecting a signature peptide of a known marker of TDP43 proteinopathy (e.g. neurofilament light polypeptide (NFL), neurofilament heavy polypeptide (NFH) and/or chitotriosidase-1 (CHIT1)).

A signature peptide of NFL may consist of the amino acid sequence of any of SEQ ID NOs: 22-28. An internal standard that is an isotope-labelled signature peptide of NFL, e.g. SEQ ID NOs: 22-24, is particularly useful with the invention.

A signature peptide of CHIT1 may consist of the amino acid sequence of any of SEQ ID NOs: 29-34. An internal standard that is an isotope-labelled signature peptide of CHIT1, e.g. SEQ ID NOs: 29-31, is particularly useful with the invention.

A signature peptide of NFH may consist of the amino acid sequence of any of SEQ ID NOs: 35-41. An internal standard that is an isotope-labelled signature peptide of NFH, e.g. SEQ ID NOs: 35-37, is particularly useful with the invention.

The internal standard precursor may also comprise a retention time standard for mass spectrometry, e.g. SEQ ID NOs: 42-51.

The internal standard precursor may comprise multiple retention time standards and/or multiple internal standards for detecting multiple signature peptides. For example, the internal standard precursor may comprise a retention time standard, an internal standard corresponding to a signature peptide of NFL, an internal standard corresponding to a signature peptide of TDP43, an internal standard corresponding to a signature peptide of NFH, and an internal standard corresponding to a signature peptide of CHIT1. The retention time standard and the internal standards may be linked in any order. For example, the internal standard precursor may consist of an isotope-labelled amino acid sequence of SEQ ID NO: 21.

Thus, the invention also provides a method of generating an internal standard precursor, comprising synthesising an internal standard corresponding to a signature peptide of TDP43 coupled to an internal standard corresponding to a signature peptide of a known marker of TDP43 proteinopathy (e.g. neurofilament light polypeptide (NFL), neurofilament heavy polypeptide (NFH) and/or chitotriosidase-1 (CHIT1). The method further comprises separating each internal standard, e.g. by enzyme digest such as chymotrypsin, before detection. The method of generating an internal standard precursor may further comprise culturing cells that are capable of expressing the internal standard precursor, wherein all sources of nitrogen for the cell culture in which the protein is synthesised contain 100% Nitrogen-15.

### Data Interpretation and Manipulation

The invention may involve determining the level of a signature peptide of a TDP-43 species, and/or a TDP-43 species that comprises a signature peptide. The method may involve a quantitative or semi-quantitative determination, or may involve a relative determination (e.g. a ratio relative to another signature peptide, or a measurement relative to the same signature peptide in a control sample).

For example, the invention may involve determining the level of a signature peptides described herein. In particular, the method may involve determining the level of a signature peptide comprising: P_{N} (SEQ ID NO: 1), P_{C} (SEQ ID NO: 2), P_{T2} (SEQ ID NO: 3) and/or P_{T3} (SEQ ID NO: 52). The invention may additionally involve determining the level of any of a signature peptide comprising SEQ ID NOs: 9, 11, 13, 15, 17 and/or 19.

The invention may involve determining the light to heavy ratio of a signature peptide described herein. In particular, the invention may involve determining the light to heavy ratio of a signature peptide comprising: P_{N} (SEQ ID NO: 1), P_{C} (SEQ ID NO: 2), P_{T2} (SEQ ID NO: 3) and/or P_{T3} (SEQ ID NO: 52). The invention may additionally involve determining the light to heavy ratio of a signature peptide comprising SEQ ID NOs: 9, 11, 13, 15, 17 and/or 19.

The invention may involve determining the ratio of the level of the signature peptide comprising P_{C} (SEQ ID NO: 2) to the level of the signature peptide comprising P_{N} (SEQ ID NO: 1). The invention may involve determining the ratio of the signature peptide comprising P_{T2} (SEQ ID NO: 3) to the level of the signature peptide comprising P_{N} (SEQ ID NO: 1). The invention may involve determining the ratio of the level of the signature peptide comprising P_{T1} (SEQ ID NO: 9) to the level of the signature peptide comprising P_{N} (SEQ ID NO: 1).

Similarly, the invention may involve determining the level of a TDP-43 species comprising a signature peptide described herein, and/or ratio between the TDP-43 species. For example, the invention may involve determining the level of a TDP-43 species comprising P_{N} (SEQ ID NO: 1), a TDP-43 species comprising P_{C} (SEQ ID NO: 2) and/or a TDP-43 species comprising P_{T2} (SEQ ID NO: 3). The invention may additionally involve determining the level of a TDP-43 species comprising any of SEQ ID NOs: 9, 11, 13, 15, 17 and/or 19. The invention may involve determining: (a) the light to heavy ratio of a TDP-43 species comprising a signature peptide, (b) the ratio of the level of a TDP-43 species comprising P_{C} (SEQ ID NO: 2) to the level of a TDP-43 species comprising P_{N} (SEQ ID NO: 1), (c) the ratio of the level of a TDP-43 species comprising P_{T2} (SEQ ID NO: 3) to the level of a TDP-43 species comprising P_{N} (SEQ ID NO: 1), and/or (d) the ratio of the level of a TDP-43 species comprising P_{T1} (SEQ ID NO: 9) to the level of a TDP-43 species comprising P_{N} (SEQ ID NO: 1).

The levels of the biomarkers (e.g. TDP-43 signature peptides) of the invention are altered in a disease cohort, compared with the control cohort. An analysis of the level of these biomarkers in the case and control populations may identify differences which provide diagnostic information. A skilled person can easily determine the relative change (e.g. up-regulation or down-regulation) for any given biomarker relative to any particular control of interest (e.g. a negative control or a positive control) in any given sample.

A control sample can be a positive control sample or a negative control sample. Typically the control sample is age-matched against the test subject. A positive control sample includes samples from confirmed cases of a condition characterised by TDP-43 proteinopathy. A negative control sample includes samples from confirmed cases of the absence of a condition characterised by TDP-43 proteinopathy. A condition characterised by non-TDP-43 proteinopathy sample can be a subject with presentation of other unrelated neurodegenerative conditions, e.g. Parkinson's disease (PD) or Alzheimer's disease (AD). The absolute levels of a biomarker in a particular control sample (e.g. samples of a subject who has PD) may be different from that in another control sample (e.g. samples of a subject who has AD). It will be appreciated that the relative expression profiles (e.g. up- or down-regulation or fold-changes) in a condition characterised by TDP-43 proteinopathy samples compared to a condition characterised by non-TDP-43 proteinopathy samples (i.e. a negative control sample) observed for the biomarkers of the invention will depend on the specific control indicated.

Usually biomarkers will be measured to provide quantitative or semi-quantitative results (whether as relative concentration, absolute concentration, fold-change, etc.) as this gives more data for use with classifier algorithms. Usually the raw data obtained from an assay for determining the presence, absence, or level (absolute or relative) requires some manipulation prior to their use. For instance, the nature of most detection techniques means that some signal will sometimes be seen even if no biomarker is actually present and so this noise may be removed before the results are interpreted. Similarly, there may be a background level of the biomarker in the general population which needs to be compensated for. Data may need scaling or standardising to facilitate inter-experiments comparisons. These and similar issues, and techniques for dealing with them, are well known in the art.

Various techniques are available to compensate for background signal in a particular experiment. For example, replicate measurements will usually be performed (e.g. using duplicate or triplicate reactions) to determine intra-assay variation and average values from the replicates can be compared (e.g. the median value of the peak area of mass spectrometry analysis). Furthermore, standard markers can be used to determine inter-assay variation and to permit calibration and/or normalisation e.g. a mass spectrometry analysis can include one or more ‘standards’, of known mass, to calibrate and to permit estimation of the unknown mass, relative to other unknown mass.

As well as compensating for variation which is inherent between different experiments, it can also be important to compensate for background levels of a biomarker which are present in the general population. Again, suitable techniques are well known. For example, levels of a particular biomarker in a sample will usually be measured quantitatively or semi-quantitatively to permit comparison to the background level of that biomarker. Various controls can be used to provide a suitable baseline for comparison, and choosing suitable controls is routine in the diagnostic field.

The measured levels of the biomarkers (e.g. a TDP-43 signature peptide), after any compensation or normalisation can be transformed into a diagnostic result respectively in various ways. This transformation may involve an algorithm which provides a diagnostic result as a function of the measured levels.

The creation of algorithms for converting measured levels or raw data into scores or results is well known in the art. For example, linear or non-linear classifier algorithms can be used. These algorithms can be trained using data from any particular technique for measuring the marker(s). Suitable training data will have been obtained by measuring the biomarkers in “case” and “control” samples i.e. samples from subjects known to suffer from a condition characterised by TDP-43 proteinopathy and from subjects known not to suffer from a condition characterised by TDP-43 proteinopathy. Most usefully the control samples will also include samples from subjects with an unrelated neurodegenerative condition, such as PD or AD, which is to be distinguished from a condition characterised by TDP-43 proteinopathy, e.g. it is useful to train the algorithm with data from subjects with prodromal and/or with data from subjects with unrelated neurodegenerative conditions. The classifier algorithm is modified until it can distinguish between the case and control samples e.g. by changing the optimal cut-off value, etc.

Thus a method of the invention may include a step of analysing biomarker levels in a subject's sample by using a classifier algorithm which distinguishes between a condition characterised by TDP-43 proteinopathy subjects and a condition characterised by non-TDP-43 proteinopathy subjects based on measured biomarker levels in samples taken from such subjects. Various suitable classifier algorithms are available e.g. linear discriminant analysis, naive Bayes classifiers, regression modelling, perceptrons, support vector machines (SVM) and genetic programming (GP), as well as a series of statistical methods such as Principal Component Analysis (PCA) and unsupervised hierarchical clustering and linear modelling.

Moreover, these approaches can potentially distinguish a condition characterised by TDP-43 proteinopathy subjects from subjects with unrelated neurodegenerative conditions. The biomarkers of the invention can be used to train such algorithms to reliably make such distinctions. The resulting data will be analysed for any potential signatures relating to differences between patient cohorts referring to levels of statistical significance (generally p<0.05), multiple testing correction and fold changes within the expression data that could be indicative of biological effect (normally it is desirable to use techniques that can indicate a change of at least 1.5 fold e.g. >1.75 fold, >2-fold, >2.5-fold, >5-fold, etc.). The classification performance (sensitivity and specificity (S+S), Receiver Operating Characteristic (ROC) analysis) of any putative biomarkers will be rigorously assessed using nested cross validation and permutation analyses prior to further validation.

### Diagnosis

A method of the invention may include a step of comparing biomarker (e.g. TDP-43 signature peptides) levels in a subject's sample to a reference. The reference may be (i) a threshold value, (ii) the corresponding biomarker level in a sample from a positive control, and/or (iii) the corresponding biomarker level in a sample from a negative control. The comparison provides a diagnostic indicator of whether the subject has the disease and/or a prognostic indicator of the severity of the disease. As would be within the understanding of a person skilled in the art, whether the level of a biomarker is increased or decreased would depend on the reference used.

Reference to the diagnosis of a subject having a condition characterised by TDP-43 proteinopathy encompasses providing a diagnostic indicator of whether the subject has amyotrophic lateral sclerosis, frontotemporal dementia or Limbic-predominant age-related TDP-43 encephalopathy. For subjects already displaying some signs and symptoms of a condition characterised by TDP-43 proteinopathy, the invention may be used to confirm or resolve another diagnosis. Thus, the invention is also useful for discriminating a condition characterised by TDP-43 proteinopathy (e.g. ALS) from a condition characterised by a proteinopathy other than TDP-43 proteinopathy (e.g. PD or AD). The invention is also useful for discriminating amyotrophic lateral sclerosis (ALS) from other forms of TDP 43 proteinopathy, e.g. Limbic-predominant age-related TDP-43 encephalopathy (LATE).

For example, in a subject having a condition characterised by TDP-43 proteinopathy, the level of P_{C} in the sample would be at a higher level than the level in a negative control sample (a sample from a subject having a condition characterised by non-TDP-43 proteinopathy, e.g. PD), and at a similar level as in a positive control sample (a sample of a subject having a condition characterised by TDP-43 proteinopathy, e.g. ALS).

By way of another example, in a subject having a condition characterised by TDP-43 proteinopathy, the level of P_{N} in the sample would be at a lower level than the level in a negative control sample (a sample from a subject having a condition characterised by non-TDP-43 proteinopathy, e.g. AD), and at a similar level as in a positive control sample (a sample of a subject having a condition characterised by TDP-43 proteinopathy, e.g. ALS).

As another example, in a subject having a condition characterised by TDP-43 proteinopathy, the ratio of the levels of P_{C} to P_{N} in the sample would be at a higher level than the level in a negative control sample (a sample from a subject having a condition characterised by non-TDP-43 proteinopathy, e.g. AD, PD, or a healthy subject), and at a similar level as in a positive control sample (a sample of a subject having a condition characterised by TDP-43 proteinopathy, e.g. ALS).

By way of another example, in a subject having a condition characterised by TDP-43 proteinopathy, the ratio of the levels of P_{T1} to P_{N} level in the sample would be at a higher level than the level in a negative control sample (a sample from a subject having a condition characterised by non-TDP-43 proteinopathy, e.g. AD, PD, or a healthy subject), and at a similar level as in a positive control sample (a sample of a subject having a condition characterised by TDP-43 proteinopathy, e.g. ALS).

By way of another example, in a subject having a condition characterised by TDP-43 proteinopathy, the level of P_{T2} in the sample may be at a lower or higher level than the level in a negative control sample (lower than a sample from a subject having AD, but higher than a subject having PD or a healthy subject), and at a similar level as in a positive control sample (a sample of a subject having a condition characterised by TDP-43 proteinopathy, e.g. ALS).

As another example, in a subject having a condition characterised by TDP-43 proteinopathy, the ratio of the levels of P_{T2} to P_{N} in the sample would be at a higher level than the level in a negative control sample (a sample from a subject having a condition characterised by non-TDP-43 proteinopathy, e.g. PD, or a healthy control), and at a similar level as in a positive control sample (a sample of a subject having a condition characterised by TDP-43 proteinopathy, e.g. ALS).

The invention may involve comparing the level of a TDP-43 signature peptide or the ratio of TDP-43 signature peptides against a threshold value, and the optimal threshold value may be determined by training classifier algorithm to distinguish between “case” and “control” samples as explained above.

For example, the reference for the light-heavy P_{N} ratio may be a threshold value of between 0.1 to 0.5, such as between 0.2 to 0.3. If a sample contains a lower light-heavy P_{N} ratio relative to the threshold value, this indicates that the subject has a condition characterised by TDP-43 proteinopathy. Conversely, if a sample contains a similar or higher light-heavy P_{N} ratio to the threshold value, this indicates that the subject does not have a condition characterised by TDP-43 proteinopathy. In the Examples below, the light-heavy P_{N} ratio under 0.29 yielded the optimal discrimination between ALS and AD, at sensitivity of 80% (CI 54.81% to 92.95%) and 87.5% specificity (CI 52.91% to 99.36%). Applying this cut-off to the CTL group, a diagnostic sensitivity of 80% (CI 54.81% to 92.95%) and specificity of 62.5% (CI 30.5% to 86.32%) was reached, and to the PD group, a sensitivity of 73% (CI 48.05% to 89.10%) and specificity of 75% (CI 40.93% to 95.56%) was obtained.

The reference for the light-heavy P_{C} ratio may be a threshold value of between 0.1 to 0.5, such as between 0.3 to 0.4. If a sample contains a higher light-heavy P_{C} ratio relative to the threshold value, this indicates that the subject has a condition characterised by TDP-43 proteinopathy. Conversely, if a sample contains a similar or lower light-heavy P_{C} ratio to the threshold value, this indicates that the subject does not have a condition characterised by TDP-43 proteinopathy. In the Examples below, the light-heavy P_{C} ratio over 0.33 yielded the optimal discrimination between ALS and PD, at sensitivity of 86.7% (CI 62.12% to 97.63%) and 100% specificity (CI 67.56% to 100.0%). Applying this cut-off to the CTL group, a diagnostic sensitivity of 86.7% (CI 62.12% to 97.63%) and specificity of 75% (CI 40.93% to 95.56%) was reached, and to the AD group, a sensitivity of 86.7% (CI 62.12% to 97.63%) and specificity of 50% (CI 21.52% to 78.48%) was obtained.

The reference for the P_{C} to P_{N} ratio may be a threshold value of between 1 to 2, such as between 1.5 and 1.6. If a sample contains a higher light-heavy P_{C} ratio relative to the threshold value, this indicates that the subject has a condition characterised by TDP-43 proteinopathy. Conversely, if a sample contains a similar or lower light-heavy P_{C} ratio to the threshold value, this indicates that the subject does not have a condition characterised by TDP-43 proteinopathy. In the Examples below, the level of P_{C} to P_{N} ratio over 1.53 yielded the optimal discrimination between ALS and CTL, at sensitivity of 100% (CI 79.61% to 100%) and 100% specificity (CI 67.56% to 100%). Applying this cut-off to the PD and AD group, a diagnostic sensitivity of 93.3% (CI 70.18% to 99.66%) and specificity of 100% (CI 67.56% to 100%) was obtained.

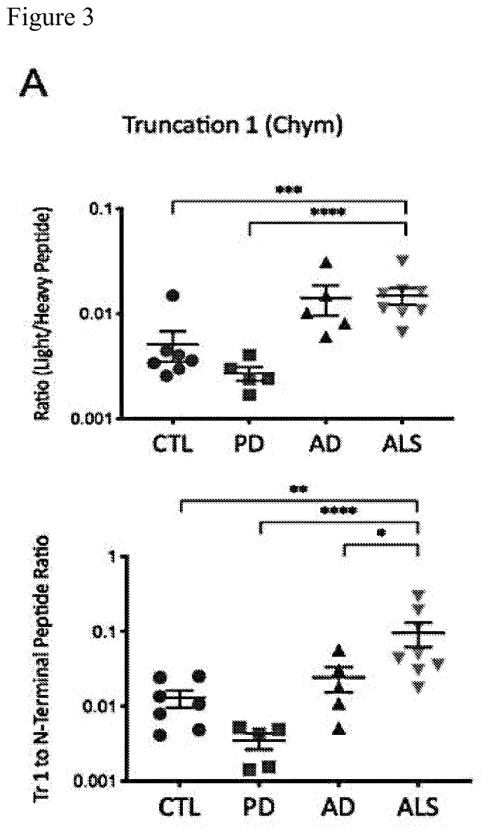

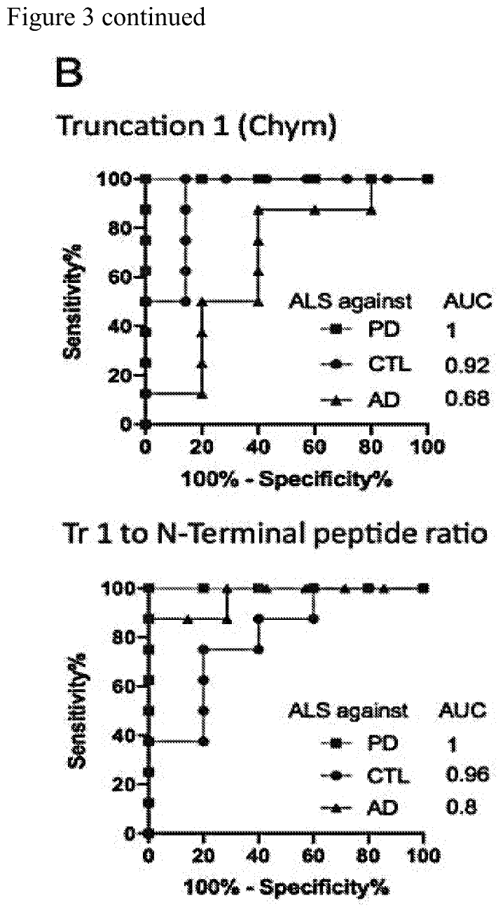

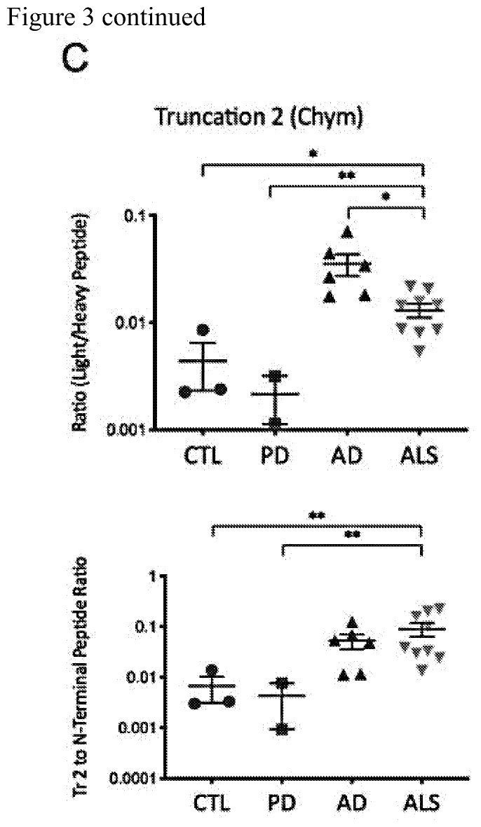

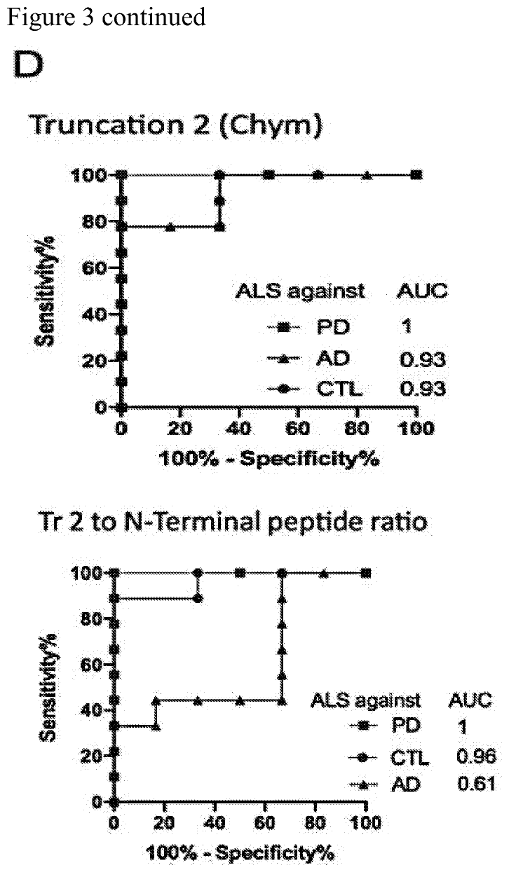

The reference for the light-heavy P_{T2} ratio may be a threshold value of between 0.01 to 0.02, such as between 0.003 to 0.005. If a sample contains a higher light-heavy P_{T2} ratio relative to the threshold value, this indicates that the subject has a condition characterised by TDP-43 proteinopathy. Conversely, if a sample contains a similar or lower light-heavy P_{C} ratio to the threshold value, this indicates that the subject does not have a condition characterised by TDP-43 proteinopathy. In the Examples below, over 0.004 yielded the optimal discrimination between ALS and PD with a sensitivity of 100% (CI 70% to 100%) and 100% specificity (CI 18% to 100%) (FIG. 3D). Applying this cut-off to the CTL group, a sensitivity of 88.89 (CI 56.50% to 99.43%) and 67% specificity (CI 11.85% to 98.29%) was obtained.

The reference for the P_{T1} to P_{N} ratio may be a threshold value of between 0.00 to 0.02, such as between 0.005 to 0.015. If a sample contains a higher level of P_{T1} to P_{N} ratio relative to the threshold value, this indicates that the subject has a condition characterised by TDP-43 proteinopathy. Conversely, if a sample contains a similar or lower light-heavy P_{C} ratio to the threshold value, this indicates that the subject does not have a condition characterised by TDP-43 proteinopathy. In the Examples below, the level of P_{T1} to P_{N} ratio with a cut-off over 0.01 achieved an optimal discrimination between ALS and AD with a sensitivity of 100% (CI 67.56% to 100.0%) and specificity of 40% (CI 7.107% to 76.93%).

Advanced statistical tools can be used to determine whether the levels determined for each biomarker in the various samples (case or control) are the same or different. For example, an in vitro diagnosis will rarely be based on comparing a single determination. Rather, an appropriate number of determinations will be made with an appropriate level of accuracy to give a desired statistical certainty with an acceptable sensitivity and/or specificity. Levels of biomarkers are measured quantitatively to permit proper comparison, and enough determinations will be made to ensure that any difference in levels can be assigned a statistical significance to a level of p<0.05 or better.

Methods of the invention may have sensitivity of at least, but not limited to, 50% (e.g. ≥50%, ≥55%, ≥60%, ≥65%, ≥70%, ≥75%, ≥80%, ≥85%, ≥90%, ≥95%, ≥96%, ≥97%, ≥98%, ≥99%).

Methods of the invention may have specificity of at least, but not limited to, 50% (e.g. ≥50%, ≥55%, ≥60%, ≥65%, ≥70%, ≥75%, ≥80%, ≥85%, ≥90%, ≥95%, ≥96%, ≥97%, ≥98%, ≥99%).

Data obtained from methods of the invention, and/or diagnostic information based on those data, may be stored in a computer medium (e.g. in RAM, in non-volatile computer memory, on CD-ROM, DVD) and/or may be transmitted between computers e.g. over the Internet.

If a method of the invention indicates that a subject has a condition characterised by TDP-43 proteinopathy, further steps may then follow. For instance, the subject may undergo confirmatory diagnostic procedures, such as those involving physical inspection of the subject, and/or may be treated with therapeutic agent(s) suitable for treating a condition characterised by TDP-43 proteinopathy. The confirmatory diagnostic procedures include known biomarkers for a condition characterised by TDP-43 proteinopathy and/or proteinopathy other than TDP-43, other information about the subject; and/or other diagnostic tests or clinical indicators for a condition characterised by TDP-43 proteinopathy. Known biomarkers for a condition characterised by TDP-43 proteinopathy includes neurofilament light polypeptide (NFL), neurofilament heavy polypeptide (NFH) and/or chitotriosidase-1 (CHIT1). Known biomarkers for a condition characterised proteinopathy other than TDP-43 includes tau and/or amyloid-B. Known diagnostic tests or clinical indicators for a condition characterised by TDP-43 proteinopathy includes muscles and imaging tests, e.g. electromyography and nerve conduction study, and/or magnetic resonance imaging (MRI) test.

### Therapy

The invention also provides a method of treating a condition characterised TDP-43 proteinopathy in a subject, comprising administering to the subject a therapy for a condition characterised by TDP-43 proteinopathy, wherein the method further comprises diagnosing if a subject has a condition characterised by TDP-43 proteinopathy according to the method of the invention, monitoring the efficacy of the therapy according to the method of the invention, and/or determining the prognosis of the disease according to the method of the invention.

Ideally treatment begins before symptom onset, i.e. before damage has occurred. The methods of the invention therefore also comprise identifying an elevated level of the TDP-43 pathological peptide signature, marking the prodromal phase of the disease. This provides an indicator of starting treatment.

A therapy for a condition characterised by TDP-43 proteinopathy, e.g. ALS, may involve administering a therapeutic agent to the subject. The therapeutic agent may be a nucleic acid molecule targeted to a gene causing TDP-43 proteinopathy, such as any of SEQ ID NO: 1-3, 7 and 52. For example, the therapeutic agent may be a nucleic acid molecule targeted to a gene causing ALS, such as an antisense oligonucleotide targeted to C9orf72. Alternatively, the therapeutic agent may be a nucleic acid molecule targeted to a gene causing FTD, such as an antisense oligonucleotide targeted to progranulin (GRN). The therapeutic agent may be a neuroprotective agent, an anti-inflammatory agent, an agent that regulates inflammation or immune system, and/or an agent that protect cells from accumulation of misfolded protein or excitatory stimuli. Examples of the therapeutic agent includes Riluzole, Endaverone, Masitinib, tauroursodeoxycholic acid, Arimoclomol, Levosimendan, asitinib, interleukin-2, and/or a Rock inhibitor e.g. Fasudil. A therapy for a condition characterised by TDP-43 proteinopathy, e.g. ALS, may involve high-caloric nutrition.

The invention also provides a nucleic acid molecule targeted to a gene causing TDP-43 proteinopathy, e.g. an antisense oligonucleotide targeted to C9orf72 or GRN, or any of SEQ ID NO: 1-3, 7 and 52, a neuroprotective agent, an anti-inflammatory agent, an agent that regulates inflammation or immune system, or an agent that protect cells from accumulation of misfolded protein or excitatory stimuli for use in a method of treating a condition characterised by TDP-43 proteinopathy in a subject, comprising administering said molecule or agent to the subject, wherein the method further comprises diagnosing if a subject has a condition characterised by TDP-43 proteinopathy according to the method of the invention, monitoring the efficacy of the therapy according to the method of the invention, and/or determining the prognosis of the disease according to the method of the invention.

The therapeutic agent may be administered intravenously, arterially, intradermally, intramuscularly, intraperitonealy, intravenously, subcutaneously, sublingually or orally (by ingestion). The compound or agent may also appropriately be introduced by rechargeable or biodegradable polymeric devices or other devices, e.g., patches and pumps, or formulations, which provide for the extended, slow, or controlled release of the compound or agent. Administering can also be performed, for example, once, a plurality of times, and/or over one or more extended periods.

### Monitoring

Methods of the invention may involve testing samples from the same subject at two or more different points in time. Methods which determine changes in biomarker(s) (e.g. TDP-43 signature peptide(s)) over time can be used, for instance, to monitor the efficacy of a therapy being administered to the subject, or to provide prognosis of the condition (i.e. indicating the likelihood of a disease or condition progression, including recurrence of a disease or condition).

Thus, the invention also provides a method of monitoring the efficacy of a therapy for a condition characterised by TDP-43 proteinopathy being administered to a subject, comprising analysing a sample from the subject according to the method of the invention. The invention also provides a method of determining the prognosis of a condition characterised by TDP-43 proteinopathy in a subject, comprising analysing a sample from the subject according to the method of the invention. In these methods, each biomarker (e.g. each TDP-43 signature peptide) of the invention may be determined according to the methods of the invention at two or more different points in time, with changing levels of each biomarker over time indicating whether the disease is getting better or worse.

The therapy may be administered before the first sample is taken, at the same time as the first sample is taken, or after the first sample is taken. The invention can be used to monitor a subject who is receiving therapy for a condition characterised by TDP-43, for example, the subject may be receiving a therapeutic agent, such as a nucleic acid molecule targeted to a gene causing TDP-43 proteinopathy, a neuroprotective agent, an antioxidant, a tyrosine kinase inhibitor and/or an inhibitor of monoamine oxidase-B, as described above.

Thus, the methods of the invention may comprise the steps of: (i) determining the level of any of signature peptide set forth in SEQ ID NOs: 1-3 and 52 in a first sample from the subject taken at a first time; and (ii) determining the level of the signature peptide in a second sample from the subject taken at a second time, wherein: (a) the second time is later than the first time; and (b) a change in the level of the signature peptide in the second sample compared with the first sample indicates that a condition characterised by TDP-43, is in remission or is progressing. Thus, the method monitors the biomarker(s) (e.g. TDP-43 signature peptide(s)) over time, with changing levels indicating whether the disease is getting better or worse. As would be within the understanding of a person skilled in the art, when the level of the biomarker changes towards the level seen in healthy controls (and away from the level seen in disease patients), the condition characterised by TDP-43 proteinopathy is in remission. On the other hand, when the level of the biomarker changes towards the level seen in disease patients or remain at the level seen in disease patients (and/or away from the level seen in healthy controls), the condition characterised by TDP-43 proteinopathy is progressing.

The disease development can be either an improvement or a worsening, and this method may be used in various ways e.g. to monitor the natural progress of a condition characterised by TDP-43 proteinopathy, or to monitor the efficacy of a therapy for a condition characterised by TDP-43 proteinopathy being administered to the subject. Thus, a subject may receive a therapeutic agent before the first time, at the first time, or between the first time and the second time.

Where the methods involve a first time and a second time, these times may differ by at least 1 day, 1 week, 1 month or 1 year. Samples may be taken regularly. The methods may involve measuring biomarkers in more than 2 samples taken at more than 2 time points i.e. there may be a 3rd sample, a 4th sample, a 5th sample, etc.

### Kit

The invention also provides diagnostic devices and kits for detecting the biomarkers of the invention. The diagnostic devices and kits of the invention may be for providing a diagnostic indicator of a subject having a condition characterised by TDP-43 proteinopathy, for discriminating a condition characterised by TDP-43 proteinopathy from a condition characterised by a proteinopathy other than TDP-43 proteinopathy, for discriminating amyotrophic lateral sclerosis from other forms of TDP 43 proteinopathy, e.g. Limbic-predominant age-related TDP-43 encephalopathy, for monitoring the efficacy of a therapy for a condition characterised by TDP-43 proteinopathy being administered to a subject, for determining the prognosis of a condition characterised by TDP-43 proteinopathy in a subject, and/or for identifying a subject susceptible to a therapy for Alzheimer's disease.

The invention also provides a diagnostic device, wherein the device comprises an internal standard or an internal standard precursor of the invention, and permits the detection of any of the signal peptides described herein in a sample.

The invention also provides a kit comprising (i) a diagnostic device of the invention and (ii) instructions for using the device to detect any of the signature peptides described herein. The kit is useful in providing a diagnostic indicator of a subject having a condition characterised by TDP-43 proteinopathy.

The invention also provides a product comprising (i) one or more internal standard or internal standard precursors of the invention which permit the detection of any of the signature peptides described herein, and (ii) a sample from a subject.

### Alternative Biomarkers

The invention has been described above by reference to TDP-43 signature peptides as biomarkers. In addition to these biomarkers, however, the invention can be used with other biological manifestations of the TDP-43 signature peptides. For example, the expression level of mRNA transcripts of the TDP-43 species comprising a signature peptide can be measured and/or further investigated.

Alternatively, the level of posttranslational modification can be determined, e.g. the phosphorylation or ubiquitination status of the TDP-43.

Further possibilities will be apparent to the skilled reader.

### Polynucleotides

The invention provides a polynucleotide encoding a fragment of TDP-43 that is 6 to 25 amino acids in length comprising an amino acid sequence having at least 90% sequence identity to the amino acid sequence of any of SEQ ID NOs: 1-3 and 52.

The invention also provides a polynucleotide encoding a fragment of TDP-43 which is 100 to 180 amino acids in length comprising an amino acid sequence having at least 90% sequence identity to the amino acid sequence of SEQ ID NO: 7.

A polynucleotide, such as a nucleic acid, is a polymer comprising two or more nucleotides. The nucleotides can be naturally occurring or artificial. A nucleotide typically contains a nucleobase, a sugar and at least one linking group, such as a phosphate, 2′O-methyl, 2′ methoxy-ethyl, phosphoramidate, methylphosphonate or phosphorothioate group. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The nucleotide is typically a ribonucleotide or deoxyribonucleotide. The nucleotide typically contains a monophosphate, diphosphate or triphosphate. Phosphates may be attached on the 5′ or 3′ side of a nucleotide.

Nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), 5-methylcytidine monophosphate, 5-methyl cytidine diphosphate, 5-methyl cytidine triphosphate, 5-hydroxymethylcytidine monophosphate, 5-hydroxymethylcytidine diphosphate, 5-hydroxymethylcytidine triphosphate, cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP), 5-methyl-2′-deoxycytidine monophosphate, 5-methyl-2′-deoxycytidine diphosphate, 5-methyl-2′-deoxycytidine triphosphate, 5-hydroxymethyl-2′-deoxycytidine monophosphate, 5-hydroxymethyl-2′-deoxycytidine diphosphate and 5-hydroxymethyl-2′-deoxycytidine triphosphate. The nucleotides are preferably selected from AMP, TMP, GMP, UMP, dAMP, dTMP, dGMP or dCMP.

The nucleotides may contain additional modifications. In particular, suitable modified nucleotides include, but are not limited to, 2′amino pyrimidines (such as 2′-amino cytidine and 2′-amino uridine), 2′-hyrdroxyl purines (such as, 2′-fluoro pyrimidines (such as 2′-fluorocytidine and 2′fluoro uridine), hydroxyl pyrimidines (such as 5′-α-P-borano uridine), 2′-O-methyl nucleotides (such as 2′-O-methyl adenosine, 2′ methyl guanosine, 2′-O-methyl cytidine and 2′-O-methyl uridine), 4′-thio pyrimidines (such as 4′-thio uridine and 4′-thio cytidine) and nucleotides have modifications of the nucleobase (such as 5-pentynyl-2′-deoxy uridine, 5-(3-aminopropyl)-uridine and 1,6-diaminohexyl-N-5-carbamoylmethyl uridine).

The nucleotides in the polynucleotide may be attached to each other in any manner. The nucleotides may be linked by phosphate, 2′O-methyl, 2′ methoxy-ethyl, phosphoramidate, methylphosphonate or phosphorothioate linkages. The nucleotides are typically attached by their sugar and phosphate groups as in nucleic acids. The nucleotides may be connected via their nucleobases as in pyrimidine dimers.

The polynucleotide can be a nucleic acid, such as deoxyribonucleic acid (DNA) or a ribonucleic acid (RNA). The polynucleotide may be any synthetic nucleic acid known in the art, such as peptide nucleic acid (PNA), glycerol nucleic acid (GNA), threose nucleic acid (TNA), locked nucleic acid (LNA), morpholino nucleic acid or other synthetic polymers with nucleotide side chains. The polynucleotide may be single stranded or double stranded.

The polynucleotide sequence may be cloned into any suitable expression vector. In an expression vector, the polynucleotide sequence encoding a construct is typically operably linked to a control sequence which is capable of providing for the expression of the coding sequence by the host cell. Such expression vectors can be used to express a construct.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Multiple copies of the same or different polynucleotide may be introduced into the vector.

The expression vector may then be introduced into a suitable host cell. Thus, a construct can be produced by inserting a polynucleotide sequence encoding a construct into an expression vector, introducing the vector into a compatible bacterial host cell, and growing the host cell under conditions which bring about expression of the polynucleotide sequence.

A polynucleotide of the invention may reduce the amount of a TDP-43 species, for example by knocking down its expression. For example, the polynucleotide may be antisense oligonucleotide. The antisense oligonucleotide may be targeted to any of SEQ ID NOs: 1-3 and 52. Antisense technology for knocking down protein expression are well known in the art and standard methods can be employed to knock down expression of a molecule of interest. Antisense oligonucleotides interfere with mRNA by binding to (hybridising with) a section of the mRNA. The antisense oligonucleotide is therefore designed to be complementary to the mRNA, although the oligonucleotide does not have to be 100% complementary (e.g. the oligonucleotide is ≥70% (at least 70%), ≥80%, ≥90%, ≥95%, ≥99% or 100% complementary to its target sequence). In other words, the antisense oligonucleotide may be a section of the cDNA. Again, the oligonucleotide sequence may not be 100% identical to the cDNA sequence (e.g. the oligonucleotide is ≥70% (at least 70%), ≥80%, ≥90%, ≥95%, ≥99% or 100% identical to the cDNA sequence. Oligonucleotides are short nucleotide polymers which typically have 50 or fewer nucleotides, such 40 or fewer, 30 or fewer, 22 or fewer, 21 or fewer, 20 or fewer, 10 or fewer or 5 or fewer nucleotides. The oligonucleotide used may be 20 to 25 nucleotides in length, more preferably 21 or 22 nucleotides in length. The nucleotides can be naturally occurring or artificial.

The polynucleotide may be a double stranded RNA, such as small interfering RNA (siRNA) or small hairpin RNA (siRNA). The double stranded RNA may be targeted to any of SEQ ID NOs: 1-3 and 52. RNA interference (RNAi) technology for knocking down protein expression are well known in the art and standard methods can be employed to knock down expression of a molecule of interest. RNAi involves the use of double-stranded RNA, such small interfering RNA (siRNA) or small hairpin RNA (shRNA), which can bind to the mRNA and inhibit protein expression.

Accordingly, the polynucleotide may comprise an oligonucleotide which specifically hybridises to a nucleic acid, e.g. mRNA, encoding a TDP-43 species. An oligonucleotide “specifically hybridises” to a target sequence when it hybridises with preferential or high affinity to the target sequence but does not substantially hybridise, does not hybridise or hybridises with only low affinity to other sequences. More preferably, the oligonucleotide hybridises to the target sequence with a Tm that is at least 5° C., at least at least 10° C., at least 20° C., at least 30° C. or at least 40° C., greater than its Tm for other nucleic acids. Conditions that permit the hybridisation are well-known in the art (for example, reference 19; and 20). The hybridisation conditions may be stringent conditions as described in the art.

### Antibody

The invention provides an antibody capable of binding specifically to a fragment of TDP-43 that is 6 to 25 amino acids in length comprising an amino acid sequence having at least 90% sequence identity to the amino acid sequence of any of SEQ ID NOs: 1-3 and 52.

The invention also provides an antibody capable of binding specifically to a fragment of TDP-43 which is 100 to 180 amino acids in length comprising an amino acid sequence having at least 90% sequence identity to the amino acid sequence of SEQ ID NO: 7.

The antibody may detect a TDP-43 species comprising the signature peptide. The antibody may bind to a TDP-43 species to decrease its function, for example by blocking its activity.

An antibody “specifically binds” to a protein when it binds with preferential or high affinity to that protein but does not substantially bind, does not bind or binds with only low affinity to other proteins. For instance, an antibody “specifically binds” a target molecule when it binds with preferential or high affinity to that target but does not substantially bind, does not bind or binds with only low affinity to other human proteins.

### Other

It is to be understood that different applications of the disclosed polypeptides and/or methods according to the invention may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural references unless the content clearly dictates otherwise. Thus, for example, reference to “a signature peptide” includes two or more “signature peptides”.

The term “comprising” encompasses “including” as well as “consisting” e.g. a composition “comprising” X may consist exclusively of X or may include something additional e.g. X+Y.

References to a “level” of a biomarker mean the amount of an analyte (e.g. a TDP-43 signature peptide) measured in a sample and this encompasses relative and absolute concentrations of the analyte, analyte titres, relationships to a threshold, rankings, percentiles, etc.

Sequence identity may be calculated using any suitable algorithm. For example the PILEUP and BLAST algorithms can be used to calculate identity or line up sequences (such as identifying equivalent or corresponding sequences (typically on their default settings), for example as described in references 21 and 22). Software for performing BLAST analyses is publicly available through the National Center for Biotechnology Information (http://www.ncbi.nlm.nih.gov/).

All publications, patents and patent applications cited herein, whether supra or infra, are hereby incorporated by reference in their entirety.

The following examples illustrate the invention.

### Example 1

The pathological hallmark of ALS is the nuclear clearance of TDP-43 in neurons and glia, with cytoplasmic inclusions of TDP-43 species, such as post-translationally modified and truncated C-terminal TDP-43 fragments. The reliable measurement of disease-specific TDP-43 species in biofluids would have potential as a biomarker of TDP-43 proteinopathies, but results from antibody-based techniques have been inconsistent to date.

The inventors aimed to identify a specific TDP-43 peptide profile for the quantification of pathological truncation of TDP-43 using liquid chromatography-tandem mass spectrometry (LC-MS/MS) combined with parallel reaction monitoring (PRM).

### Methods and Materials

### Human Samples

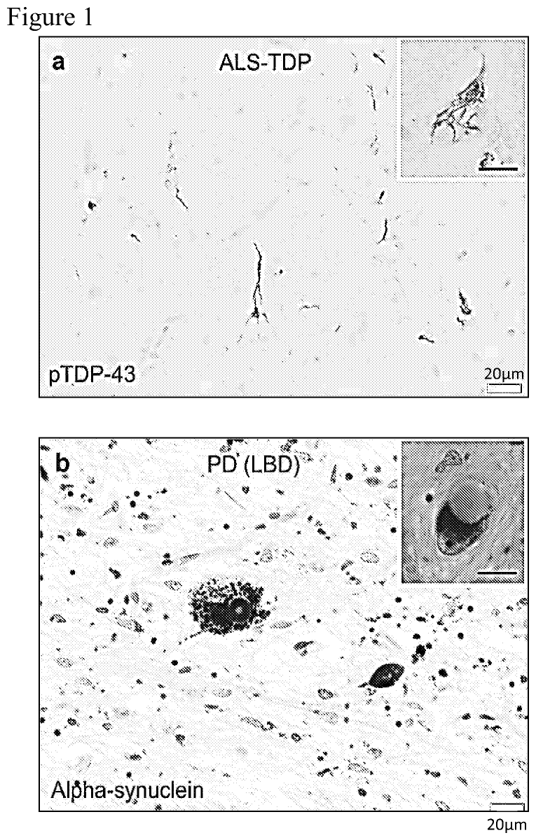

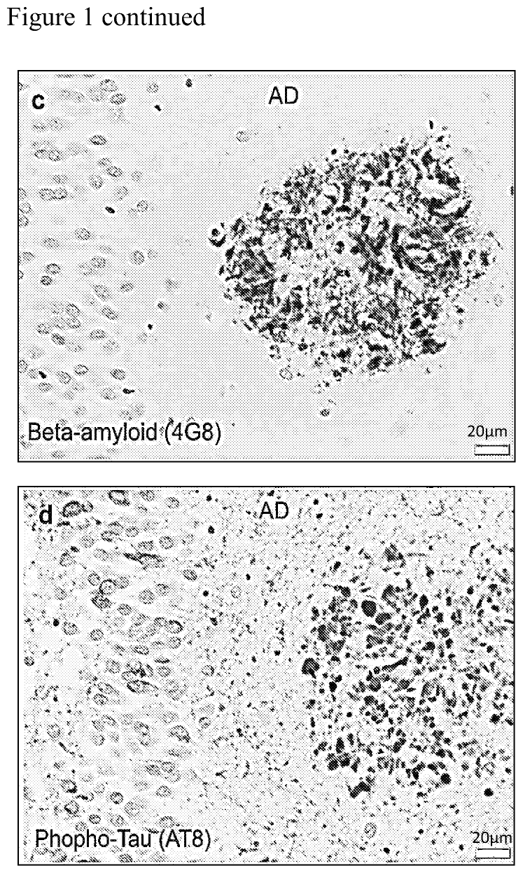

Cortex and spinal cord tissues were obtained from healthy controls (CTL), subjects having Parkinson's disease (PD), subjects having Alzheimer's disease (AD) and subjects having amyotrophic lateral sclerosis (ALS). Characteristics of the tissue cohort are provided in Table 1. The AD group was significantly older than CTL, ALS and PD (p=0.0005). All clinical cases were neuropathologically confirmed and the characteristic neuropathology of each diagnostic group is shown in FIG. 1.

TABLE 1
Characteristics of post mortem tissue cohort

| | Cortex, n | Spinal Cord, n | | Gender |
|---|---|---|---|---|
| Diagnostic | (Motor/ | (Thoracic/ | Mean age | (female/ |
| groups | Frontal) | Cervical/Lumbar) | (years) | male) |
| CTL | 8 (8/0) | 8 (7/1/0) | 65.9 | (4/4) |
| PD | 8 (5/3) | 6 (6/0/0) | 76.9 | (1/7) |
| AD | 8 (8/0) | 8 (8/0/0) | 84.3*** | (4/4) |
| ALS | 16 (14/3) | 16 (12/3/1) | 65.6 | (6/10) |

***significantly increased to all other groups (p < 0.001)

### Immunohistochemistry for Microscopic Analysis of Neurodegenerative Proteinopathies

All cases were formally assessed for microscopically visible protein aggregates using standard criteria used in international brain banking (PMC3266529). In brief, formalin-fixed paraffin-embedded sections (six microns) were stained with the following primary antibodies (source, dilution and antigen retrieval method in parentheses): pTDP-43 (Cosmobio, 1:20,000, autoclave—citrate buffer); Anti-B-Amyloid (4G8—Biolegend, 1; 24,000, formic acid); AT8 (Innogenetics, 1:1500, none); Purified Mouse Anti-a-Synuclein (BD Laboratories, 1:1000, formic acid). All were developed with the DAKO Envision Kit and 3,3-diaminobenzidine tetrahydrochloride (DAB) as chromogen. Sections were examined using an Olympus BX50 microscope with an attached Olympus digital camera (for photography).

Extraction of Soluble and Insoluble Protein Fractions from Tissue

Fractionation of tissue was done as described in reference 1 with the following modifications. Frozen tissues from cortex and spinal cord of all groups of subjects were homogenized in 1:1 volume:tissue weight low-salt homogenization-solubilization (LS) buffer (10 mM Tris pH8.0, 5 mM EDTA, 1 mM dithiothreitol) supplemented with protease and phosphatase inhibitors (Thermo Scientific), 0.2 mM Sodium orthovanadate and 0.3 Unit/μl benzonase nuclease. Cortex and spinal cord samples were homogenized using steel beads in a TissueLyser® system (Qiagen) and centrifuged. The pellets were homogenized in Triton-X (TX) buffer (1% Triton X-100, 0.5M NaCl in LS buffer).

All samples were equalized to 20 mg of total protein in a final volume of 3 ml Triton-X buffer. Tissue homogenates were centrifuged, and the supernatants, corresponding to the soluble proteins, were saved as the Triton-X fraction (TF). Pellets were washed and subsequently resuspended in sarkosyl buffer (1% N-lauroyl-sarcosine, 0.5M NaCl in LS buffer), briefly sonicated, and incubated at 37° C. for 30 minutes followed by centrifugation. Supernatants were saved as the sarkosyl fraction (SF), while the pellets were resuspended in urea buffer (7M urea, 2M Thiourea, 4% CHAPS, 30 mM Tris pH8) supplemented with protease and phosphatase inhibitors (Thermo Scientific). Samples were sonicated and centrifuged, and the supernatant, corresponding to the insoluble fraction, was saved as the urea-soluble fraction (UF) and frozen at −80° C.

### Insoluble Fractions Preparation for Mass Spectrometry Analysis

Urea fractions (UF) were thawed and reduced by incubation with 5 mM dithiothreitol, followed by alkylation by incubation with 20 mM iodoacetic acid (IAA). Samples were subsequently precipitated by methanol-chloroform extraction. Pellets were fully resuspended by addition of 6M Urea and brief sonication. 50 mM TEAB was subsequently added to reduce the final concentration of urea to 1M. Half the volume of the samples were incubated with trypsin at 37° C. for 14-16 h, while the other half was incubated with chymotrypsin at 22° C. for 14-16 h. The reaction was stopped by addition of formic acid. Samples were desalted using SOLA SPE columns (ThermoFisher), dried in a speed-vac (ThermoScientific) and pellets were resuspended in 2% acetonitrile with 0.1% formic acid diluted in MilliQ water. The peptide amount per sample was measured using a quantitative colorimetric peptide assay (Pierce) as per manufacturer's protocol.

### Discovery Proteomics

Peptides from in-solution and in-gel digestion were analysed by nano ultra-high-performance liquid chromatography tandem mass spectrometry (nano-UPLC-MS/MS) using a Dionex Ultimate 3000 nano-UPLC, (Thermo Scientific) coupled to an Orbitrap Fusion Lumos Tribrid mass spectrometer (Thermo Scientific). Data were acquired in data dependent mode with a resolution of 120,000 fullwidth half maximum at m/z 200 in the survey scan (375-1500 m/z) and with EASY-IC using the reagent ion source (202 m/z) for internal calibration. MS/MS spectra were acquired after precursor isolation in the quadrupole with an isolation window of 1.2Th, dynamic precursor selection (top speed mode) with a 3-second fixed duty cycle time and 60-second dynamic precursor exclusion. Isolated precursor ions were fragmented by CID with a Normalised Collision Energy of 35%.

Parallelization was enabled and MS/MS spectra were acquired in the linear ion trap for up to 250 ms with an ion target of 4000 in rapid scan mode. Raw MS data were analysed using Progenesis QI for Proteomics v3.0 (Nonlinear Dynamics). MS/MS spectra were searched against the UniProt Homo sapiens Reference proteome (retrieved Nov. 15, 2016) using PEAKS with precursor mass tolerance of 10 ppm and a fragment ion tolerance of 0.5 Da. Carbamidomethylation of Cysteines was defined as a fixed modification, and deamidation of Asparagine and Glutamine and oxidation of Methionine as variable modifications. Peptides scoring ≥20 and FDR<1% were imported into Progenesis QIP.

### Generation of Heavy Isotope-Labelled Peptides

Heavy isotope-labelled, AQUA-grade peptides (heavy peptides) (ThermoFisher) were used for the relative and absolute quantitation of endogenous proteins. Heavy peptides were commercially synthesized using Fmoc solid-phase technology and purified by HPLC as per manufacturer's protocol (ThermoFisher). AQUA-grade peptides are delivered in solution with a concentration of 5 μmol/μl per peptide.

### Parallel Reaction Monitoring (PRM)

1 μl of trypsin- or chymotrypsin-digested and desalted samples were injected into the mass spectrometer. A time schedule targeted MS/MS method was used with peptide-specific parameters as described in Table 2. 10 fmol of the heavy isotope labelled peptides listed in Table 2 were injected and as technical quality check pools of samples for each group were prepared and loaded every 10th run to determine the reproducibility of the assay.

MS data processing was conducted using the software package Skyline. Very briefly, Skyline was used to export ion chromatogram data for the light and heavy versions of each peptide in each analysis run. Then the elution peaks of each light and heavy peptide pair were located in each sample. Pairs in each sample were filtered to remove those with insufficient signal for quantitation (minimum 2 monitorable fragment ion values measured over at least 3 collected spectra) or poor-quality data (pearson correlation between light and heavy chromatogram data less than 0.5). Elution peak boundaries were assigned at the point where the heavy peptide signal dipped below 1% of its max intensity in that sample. Finally, a light-heavy ratio was calculated for each peptide by finding the gradient of a fitted simple linear model with no intercept (L=mH) to the light and heavy data points at each time point.

The absolute amount (fmol) of each peptide was calculated by dividing the peptide ratio by 10 according to the concentration of the heavy peptide (10 fmol).

### Western Blotting

The TX and UF samples were diluted in LDS sample buffer (ThermoFisher) supplemented with reducing agents (ThermoFisher) and boiled for 5 minutes. Proteins were separated on 10% or 4-12% NuPAGE pre-cast Bis-Tris gels (ThermoFisher) and transferred using an iBlot2 apparatus (ThermoFisher) after ponceau whole protein staining. Membrane were blocked using protein free Blocking buffer (ThermoFisher) and probed over-night at 4° C. with primary antibodies: TDP-43 C-terminus (Proteintech). Membranes were incubated at room temperature for 2 hrs with secondary antibody rabbit HRP. Antibody signal was detected with ECL reagent (GEHealthcare) using a ChemiDoc MP Imaging System (Bio-Rad). For interpretation of the immunoblots the ratios of the intensities of the C-terminal fragments over full-length TDP-43 from ALS were compared against all other diseases using one-way ANOVA and Dunnett's multiple comparison test.

### Silver Staining

Total protein amounts were visualised by silver staining using a ProteoSilver Silver Stain kit (Sigma) as per the manufacturer's protocol and using solutions provided in the kit. Briefly, after electrophoresis, NuPAGE Bis-tris gels were fixed overnight at room temperature in fixing solution (10% acetic acid, 50% Ethanol in MilliQ water). The gels were incubated in 30% Ethanol, MilliQ water, sensitizer solution, and then silver solution. Staining was visualised by incubating with developer solution, the reaction was stopped by the addition of ProteoSilver Stop solution followed by washing.

### Statistical Analysis

For interpretation of the parallel reaction monitoring results across diagnostic groups, the absolute abundances of the light peptide (log 10 ratio (light/heavy peptide) were compared by testing significant differences between ALS to all other diagnostic groups using one-way ANOVA with Dunnett's multiple comparison test. Standard measures of diagnostic test validity, such as sensitivity, specificity and predictive values accompanied by their CIs stated at the 95% confidence level, were calculated for varying peptide cut-off levels. The optimal cut-off level for dichotomising values was selected as the situation maximising the Youden index (23). The receiver operating characteristics (ROC) curve is used for a graphical visualisation of the impact of the variation in the cut-off values.

### Results

### Discovery and Validation of TDP-43 Peptides in Brain Urea Fractions

Cortex tissues from 2 separate ALS patients and CTLs were used for the discovery of TDP-43 peptides by bottom-up mass spectrometry. After enrichment of the insoluble protein fraction, TDP-43 was detected by LC-MS/MS analysis from both ALS (n=2) and healthy control (n=2) urea fractions. In-solution digestion with trypsin and chymotrypsin allowed the detection of 4 enzyme specific TDP-43 peptides that sufficiently covered the N-terminal (SEQ ID NOs: 1 and 11) and C-terminal end of TDP-43 (SEQ ID NOs: 2 and 19), as shown in Table 2. The locations of these peptides within TDP-43 are indicated in FIG. 7.

In addition, in-gel digestion of low molecular weight bands (23-28 kDa) of ALS urea fractions allowed the identification of semi-specific TDP-43 peptides (SEQ ID NOs: 3, 9, 15 and 17). The locations of these peptides within TDP-43 are indicated in FIG. 7. These peptides include one cleavage site independent of the enzyme used for digestion (non-specific digestion pattern) which suggests that truncation of TDP-43 has occurred endogenously. Semi-specific peptides are therefore able to represent a specific truncation site of low molecular weight TDP-43 fragments. N-terminal nonspecific peptides (SEQ ID NOs: 3, 9 and 17) have a N-Terminal endogenous truncation site and cover a C-terminal TDP-43 amino acid sequence that represent a low molecular weight C-terminal TDP-43 fragment.

For the measurement of endogenous (light) TDP-43 peptides by parallel reaction monitoring, C- and N-Terminal and truncation site specific peptides detected from the discovery experiments, in silico predicted and previously observed peptides were used to generate heavy isotope-labelled peptides (SEQ ID NOs: 4-6, 10, 12, 14, 16 18 and 20). Properties of all peptides for parallel reaction monitoring and selected peptides used for the final quantification across diagnostic groups are given in Table 2.

Final peptides used for peptide validation across diagnostic groups were selected based on their technical reliability including a small chromatographic point shift and divergence of the light and heavy peptide for accurate quantification (selected peptides for quantification across diagnostic groups: SEQ ID NOs: 4-6, 10). Pre-analytical sample screening prior to TDP-43 quantification by parallel reaction monitoring included silver stains of all soluble (TX) and final insoluble (urea) protein fractions were performed at equal volumes to verify the recovery of protein prior to digestion.

The C- to N-Terminal TDP-43 Peptide Ratio is Increased in Urea Fractions from ALS Brain Tissue and Discriminates Between ALS from Other Neurodegenerative Diseases

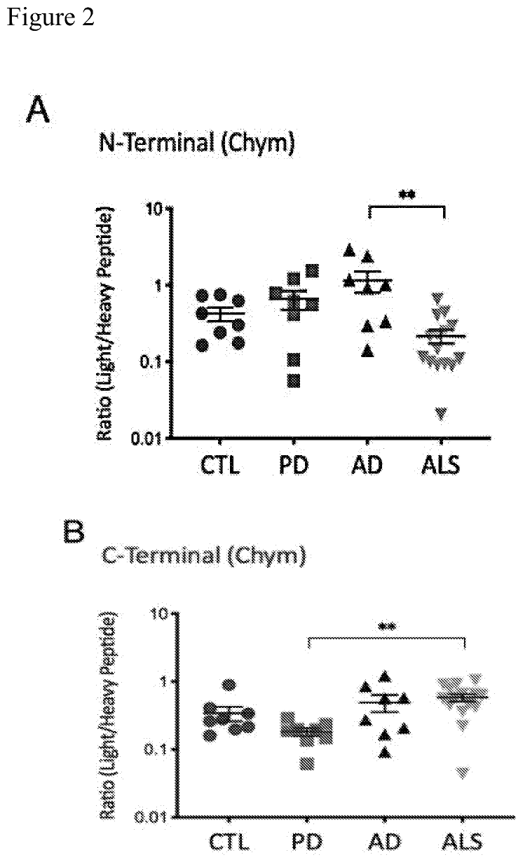

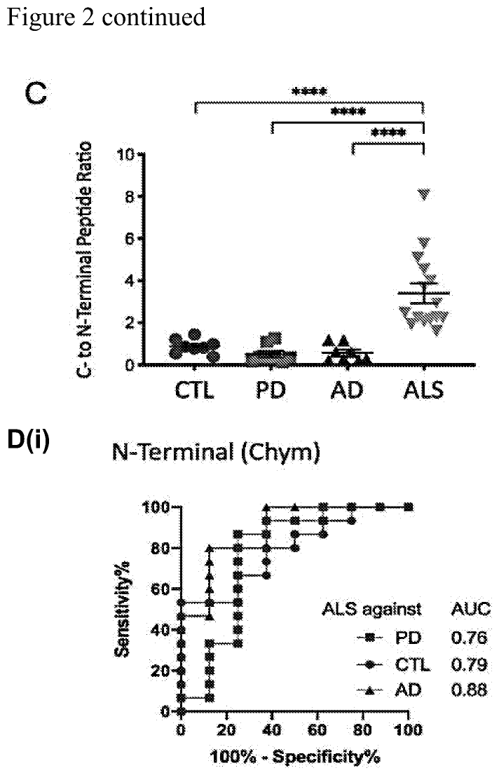

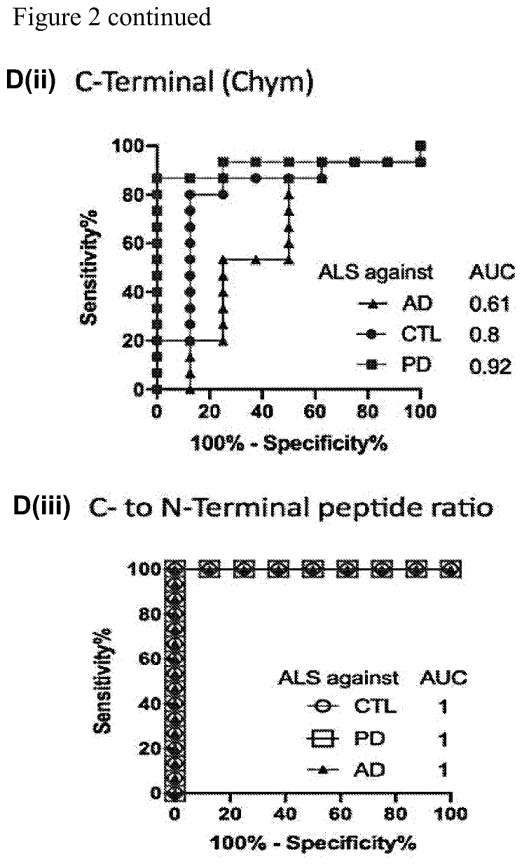

Quantification of the individual peptide ratios (light peptide:heavy peptide) showed that the chymotryptic N-terminal peptide ratio was lower in ALS compared to AD (p=0.001) (FIG. 2a). In contrast, the chymotryptic C-terminal peptide ratio was increased in ALS compared to PD (p=0.005) (FIG. 2b), but unaltered between ALS and CTL or AD. TDP-43 pathology in ALS brains is characterized by an increase in C-terminal TDP-43 fragments (1,7). To investigate, if the measurement of the peptide ratios could reflect this pathology, the C:N-terminal peptide ratio was calculated. The C:N-terminal peptide ratio was increased in ALS compared to all other diagnostic groups (p=0.0001 respectively) (FIG. 2c). This suggests that the C:N-terminal ratio is a highly specific marker of ALS representing the enrichment of C-terminal TDP-43 protein fragments and/or a relative lack of N-terminal TDP-43 in the insoluble protein fractions.

To determine the potential of using C- and N-terminal peptide quantification as a diagnostic tool to discriminate ALS from other diseases, receiver operating characteristic (ROC) analysis was used (FIG. 2d). N-terminal light:heavy peptide <0.3 yielded the optimal discrimination between ALS and AD, with 80% (CI 54.8-93.0) sensitivity and 87.5% (CI 52.9-99.4) specificity. Applying this cut-off to the CTL group gave a diagnostic sensitivity of 80% (CI 54.8-93.0) and specificity of 62.5% (CI 30.5-86.3), and for the PD group a sensitivity of 73% (CI 48.1-89.1) and specificity of 75% (CI 40.9-95.6). C-terminal light:heavy peptide >0.3 yielded the optimal discrimination between ALS and PD, with a sensitivity of 86.7% (CI 62.1-97.6) and 100% (CI 67.6-100) specificity. Applying this cut-off to the CTL group gave a diagnostic sensitivity of 86.7% (CI 62.1-97.6) and specificity of 75% (CI 40.9-95.6), and to the AD group, a sensitivity of 86.7% (CI 62.1-97.6) and specificity of 50% (CI 21.5-78.5). A C:N-terminal peptide ratio >1.5 yielded the optimal discrimination between ALS and CTL, with a sensitivity of 100% (CI 79.6-100) and 100% specificity (CI 67.6-100). Applying this cut-off to the PD and AD group gave a diagnostic sensitivity of 93.3% (CI 70.2-99.7) and specificity of 100% (CI 67.6-100).

### Truncation Site-Specific Peptides Detect Pathological TDP-43 Processing in ALS and AD

The truncation site-specific peptides measure a specific N-terminal cleavage site and therefore have the potential to specifically quantify pathological processing of TDP-43 (24). Quantification of the peptide ratio (light:heavy) of the first chymotryptic truncation site-specific peptide 1, which was previously observed by Kametani (24), showed an increase in ALS cortex urea fractions compared to PD and CTL (p=0.0008 and p<0.0001 respectively). Only the Truncation 1:N-terminal peptide ratio showed an increase in ALS compared to AD due to the respective higher N-terminal peptide ratio in AD (p=0.04) (FIG. 3a).

ROC analysis showed that a Truncation 1 peptide ratio >0.005 yielded the optimal discrimination between ALS and PD, with a sensitivity of 100% (CI 67.56% to 100.0%) and 100% specificity (CI 56.55% to 100.0%) (FIG. 3b). Applying this cut-off to the CTL group gave a sensitivity of 100% (CI 67.6-100) with 85.7% specificity (CI 48.7-99.3). The same cut-off value applied to the AD group showed a sensitivity of 100% (CI 67.6-100.0), but a low specificity of 20% (CI 1.0-62.5). The Truncation 1:N-terminal peptide ratio >0.01 achieved a better discrimination between ALS and AD with a sensitivity of 100% (CI 67.6-100.0) and specificity of 40% (CI 7.1-76.9).

A new N-terminal truncation site was detected in the discovery experiments and reflects a shorter C-terminal fragment than Truncation 1. Because the Truncation 2 peptide contains a chymotryptic cleavage site, endogenous peptide is only produced by (relatively less frequent) incomplete digestion with chymotrypsin; a so-called ‘missed cleavage’ after the position 5 leucine residue. The alternative sequence produced after complete cleavage was too short to make monitoring by PRM plausible. The yield of the missed-cleavage containing peptide from digestion could be assumed to be substantial since the peptide had been observed in the discovery experiment, so the peptide was explored despite this issue. Because the peptide is generated by digestion according to an (unknown) site-specific missed cleavage rate, there was therefore a relatively lower yield of this peptide after digestion in comparison to the other peptides, and the mass spectrometer acquisition method was adapted to allow for this. The statistical analysis assumes that there was no significant variation in the site-specific missed cleavage rate of chymotrypsin (and therefore final peptide yield) between samples. Quantification of the new chymotryptic truncation site specific peptide 2 showed a significant increase in ALS compared to PD and CTL (p=0.001 and p=0.01 respectively) (FIG. 3c), but Truncation 2 was increased in AD as compared to ALS (p=0.01). No significant difference between ALS and AD was observed when calculating the Truncation 2:N-terminal peptide ratio (p=0.79). According to this, ROC analysis demonstrated a Truncation 2 peptide ratio >0.004 yielded the optimal discrimination between ALS and PD with a sensitivity of 100% (CI 70-100) and 100% specificity (CI 18-100) (FIG. 3d). Applying this cut-off to the CTL group gave a sensitivity of 88.9% (CI 56.5-99.4) and specificity of 67% (CI 11.9-98.3). However, for ALS and AD only a cut-off value below 0.016 discriminated between both with a sensitivity of 77.8% (CI 45.3-96.1) and specificity of 100% (CI 61.0-100). When the Truncation 2:N-terminal peptide ratio was calculated discrimination between AD and ALS decreased with an AUC of 0.61. This data shows that an increase of truncation site specific peptides occurs both in ALS and AD and are not sufficient to discriminate between ALS and AD.

C-Terminal TDP-43 Fragments Accumulate in the Insoluble Protein Fraction of the Cortex of Patients with ALS

To confirm the proteomic findings, soluble (TX) and insoluble (urea) protein fractions from the same cortex tissue cohort of ALS, CTL, PD and AD used for parallel reaction monitoring were immunoblotted with anti-C-Terminal TDP-43 antibody to determine the ratio of C-Terminal lower molecular weight bands to full-length TDP-43. Immunoblotting of the proteins soluble in the high-salt TX fraction shows that TDP-43 is present under various forms. When using an antibody specific for C-Terminal TDP-43, lower molecular weight bands at 35 kDa and 25 kDa were present suggesting C-Terminal fragments in addition to full-length TDP-43 at 43 kDa in accordance with previous observations (1). Larger TDP-43 forms (around 55 kDa) were also present, likely representing post-translationally modified TDP-43. Importantly, the various forms of TDP-43 appeared to be present in the CTL, ALS, PD and AD samples (FIG. 4A). Given that C-Terminal fragments (CTF) at 35 kDa and 25 kDa have been previously suggested as associated with ALS (31), the ratio of CTF-35 or CTF-25 levels over full-length TDP-43 levels was calculated. The CTF-25/TDP-43 and CTF-35/TDP-43 ratios were not significantly increased across diagnostic groups (FIG. 4A). Therefore, CTF-25 and CTF-35 do not seem to be associated with disease when present in the soluble protein fraction. However, when similar experiments and quantifications were performed on the insoluble (urea) protein fractions, a significant increase in the level of CTF-25 over the level of TDP-43 was observed in the cortex of ALS patients in accordance with the proteomics data (FIG. 4B). Noteworthy, CTF-25 and CTF-35 fragments were also detected in the cortex of AD patients (FIG. 4B), however without a significant increase of the CTF to full-length TDP-43 ratios. Together, this data shows that increased CTF-25 present in the insoluble protein fraction is associated with ALS and that CTFs are present in AD, however at similar levels of full-length TDP-43.

Pathological TDP-43 is Present in the Cortex of AD Patients with Increased Truncation Site Specific Peptides

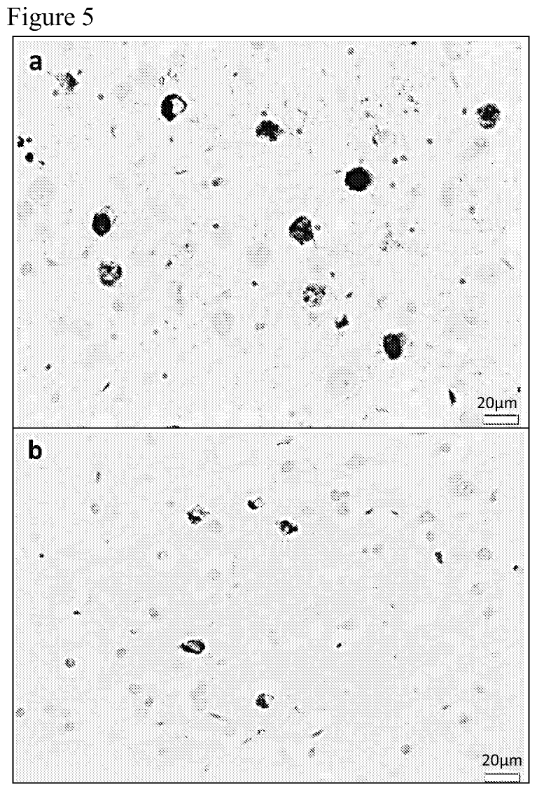

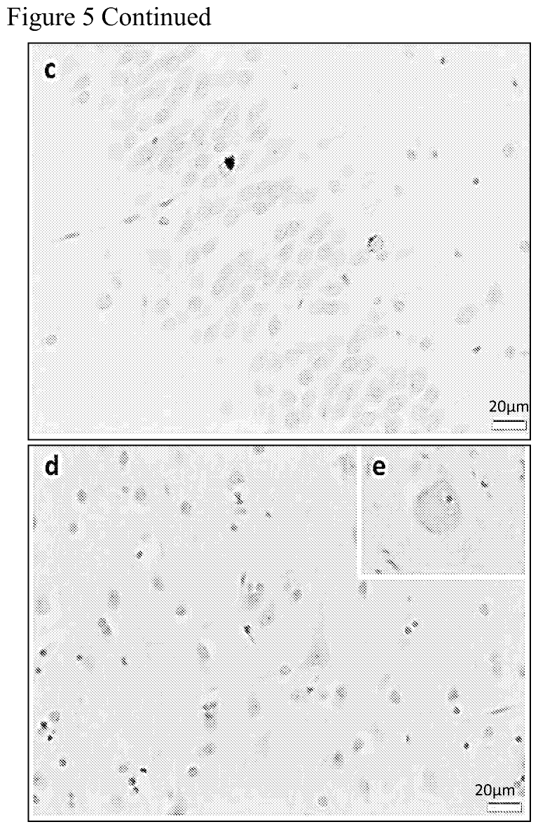

Due to pathological TDP-43 truncation found in AD cases by PRM, it was investigated, if LATE may coexist in significant cases of AD. Therefore, the cohort for microscopic evidence of pTDP-43 pathology was re-examined. It was found that there is evidence of LATE in six out of eight cases of AD, but not in PD, ALS or controls. The principal anatomical distribution of pTDP-43 in LATE was different from that typically found in ALS, mainly involving the amygdala, entorhinal cortex and hippocampus (FIG. 5 and Table 3).

TABLE 3
AD phosphoTDP-43 staging

| | M1 | | | Hippocampus | |
|---|---|---|---|---|---|
| | (Motor | Medulla | | Granule cell | Temporal |
| Diagnosis | Cortex) | oblongata | Amygdala | layer | Cortex/Er |
| AD | − | − | + | − | − |
| AD | − | − | + | − | + |
| AD | − | − | + | − | ++ |
| AD | − | − | +++ | + | +++ |
| AD | − | − | − | − | − |
| AD | − | − | − | − | − |
| AD | + | − | +++ | + | +++ |
| AD | − | − | + | − | + |

### C-Terminal TDP-43 is not Increased in the Urea Fraction of Spinal Cord

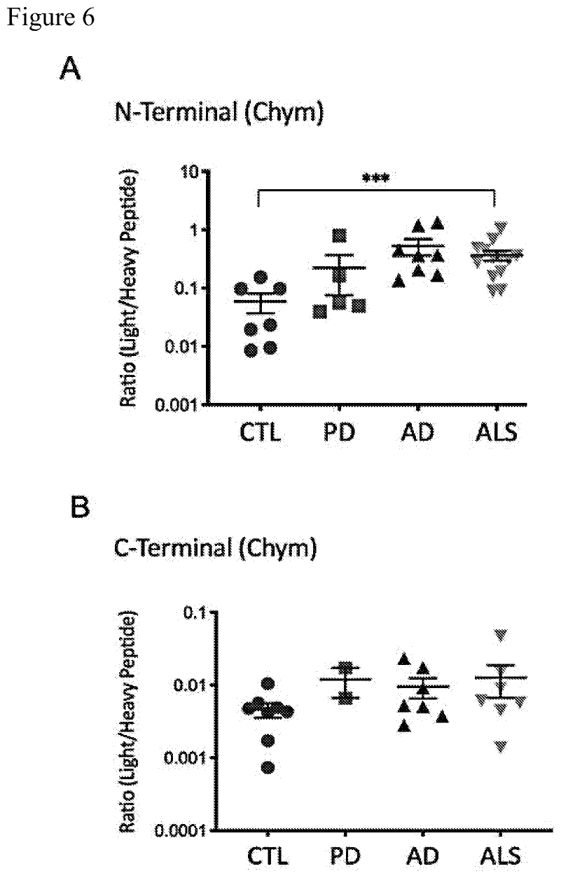

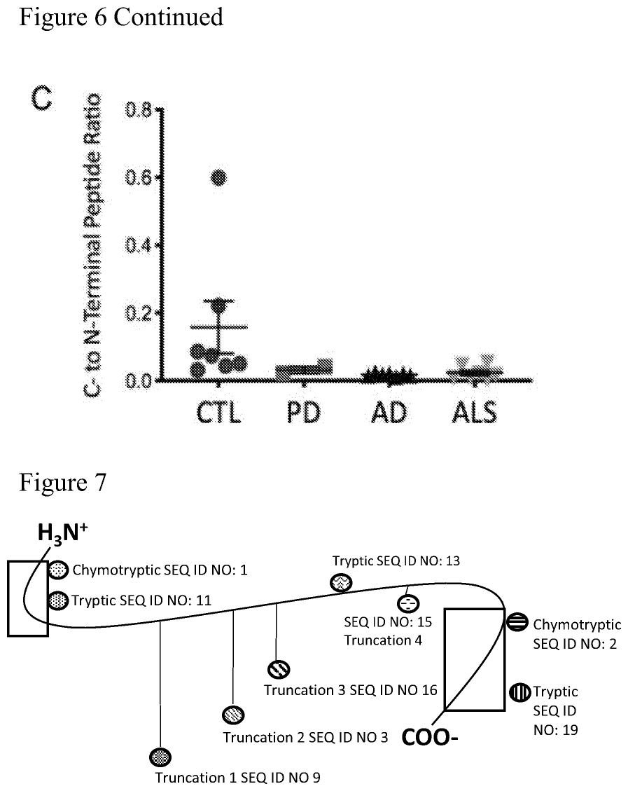

To investigate differences of TDP-43 pathology in affected regions of ALS, case matched spinal cord samples were investigated. In contrast to data from the cortex, quantification of the N-terminal light:heavy peptide ratio in spinal cord urea samples showed an increase in ALS compared to CTL (p=0.0001) (FIG. 6a). The chymotryptic C-terminal peptide ratio was unaltered in ALS as compared to CTL (p=0.3), PD or AD (both p=0.9) (FIG. 6b). This suggests the presence of N-terminal TDP-43 in spinal cord urea fractions compared to cortex urea fractions, in line with a previous immunohistochemistry analysis, which shows that cytoplasmic inclusions in the spinal cord stain with both a C-terminal TDP-43 and N-terminal TDP-43 antibody (5). When calculating the C:N-terminal peptide ratio to assess the proportion of C-terminal versus full-length TDP-43, a reverse pattern compared to cortex ALS urea fractions was observed, with an unaltered C:N-terminal peptide ratio when compared to CTL (p=0.1) (FIG. 6c). Truncation site-specific peptide 1 was not detected in any of the samples regardless of the diagnostic groups while Truncation 2 was only detected in 2 CTL, 1 PD and 1 AD sample.

### Discussion

Previous studies measuring TDP-43 in CSF and serum using antibody-based methods have revealed inconsistent results due to the non-specific binding of antibodies to full-length and pathologically altered TDP-43 (8,12). To overcome this problem, the inventors demonstrated that TDP-43 from brain tissues can be detected by mass spectrometry and discovered potentially pathognomonic TDP-43 peptides. A sophisticated targeted proteomic methodology was then used to validate and quantify TDP-43 peptides across an independent post mortem tissue cohort for the identification of an ALS-specific peptide signature. This included peptides that were located at the N-terminus, a peptide region that is also recognized by an antibody specifically detecting full-length TDP-43 (13), and the C-terminus of TDP-43 found in pathological TDP-43 aggregates from ALS brains (7). Truncation peptides, which have the potential to reflect N-terminal cleavage of C-terminal fragments (24), were quantified across diagnostic groups to assess their disease-specificity. This combined peptide approach showed that an increased C:N-terminal peptide ratio is a sensitive and highly specific marker for ALS, which is consistent with decreased levels of full-length TDP-43 and the accumulation of C-terminal TDP-43 in urea fractions of ALS brain tissue, supported by previously published data using a different approach to isolate pathological TDP-43 (6). The data, showing an increase of truncation site-specific peptides in ALS also support N-terminal truncation as the disease-specific mechanism for C-terminal TDP-43 accumulation.

The pathological basis for the novel truncation site is unclear, as previously described reactions such as calpain- or caspase-cleavage to generate fragments of the size of 25 kDa and 35 kDa do not occur on the relevant amino acid site (25,26). The presence of truncation sites in AD suggests that pathological TDP-43 processing occurs in both diseases. However, the normal C:N-terminal peptide ratio in AD suggests that truncation of TDP-43 does not occur to a sufficient level to result in C-terminal fragment accumulation. However, more detailed neuropathological analysis confirmed that in most AD cases TDP-43 pathology existed in addition to predominant Tau and Amyloid-B pathology. This suggests, that the new method is highly sensitive in detecting biochemical changes of pathological TDP-43 processing, even before the typical neuropathological manifestations of TDP-43 pathology occur.

This study confirmed previous immunohistochemical findings that cytoplasmic TDP-43 inclusions within the brain stain for C- but not N-terminal TDP-43, whereas ALS spinal cord TDP-43 inclusions are positive for both (7). Whether this comes from alternative truncation of TDP-43, as evidenced by the lack of detection of truncation site-specific peptides in spinal cord, or as a result of different clearance mechanisms between cortical and spinal motor neurons, is unclear (27,28,29,30). Further investigation of differential pathological processing of TDP-43 in relation to different neuroanatomical regions is needed (31).

In summary, the detection of a specific peptide signature enables the quantification of pathological C-terminal fragment accumulation of TDP-43 in post mortem ALS brain tissue allowing discrimination from other neurodegenerative diseases. This offers the opportunity to develop a disease-specific ex vivo assay for ALS, which would have major significant potential as a diagnostic and pharmacodynamic biomarker, if such a signature can be detected in accessible biofluids such as serum and CSF. Furthermore, the data suggest that this peptide signature could form the basis for stratification of AD according to TDP-43 pathology, which might have significant implications for therapeutic trial design.

### Example 2

It was investigated whether the disease-specific TDP-43 species can be identified from cerebrospinal fluid (CSF) samples using mass spectrometry.

In this experiment, two samples were compared: (1) unprocessed, native CSF samples (CSF samples) and (2) processed CSF samples containing extracellular vesicles (EV) (EV-containing samples). The samples were subjected to nano-UPLC-MS/MS, combined with RPM, as set out in Example 1.

2×500 μl of 16 CSF samples from clinically-well characterised ALS patients were taken.

EV-containing samples were obtained in the following way. Samples were combined and underwent centrifugation, filtration, ultrafiltration, washing and centrifugation. The retentate was injected into a size exclusion column packed with sepharose 4 fastflow (mean particle size 90 μm, exclusion limit 3×10⁷) and eluted with 40 mL PBS at 0.5 mL min⁻¹ using an ÄKTA pure chromatography system (GE Life Sciences). Two microliter fractions were collected from 6 to 40 mL elution volume. Extracellular vesicle (EV)-containing fractions (2-3) were concentrated for further analysis.

The CSF and EV-containing samples were combined and processed for mass spectrometry. The samples were reduced in 5 mm dithiothreitol for 30 min at room temperature followed by alkylation with 20 mm iodoacetamide for 30 min at room temperature and precipitated using chloroform-methanol precipitation. Precipitated protein was resuspended in 50 mm triethylammonium bicarbonate. Samples were digested overnight at 37° C. with 300 rpm shaking using 400 ng of chymotrypsin for EV-containing samples and a 1:50 trypsin:protein ratio for CSF samples. Peptide digests were acidified with 1% formic acid, desalted using Sep-Pak C18 cartridges (Waters), and dried by vacuum centrifugation. Peptides from EV-containing samples were resuspended in 10 μL buffer A (2% acetonitrile, 0.1% formic acid in water); CSF samples were resuspended to a final concentration of 500 ng μL⁻¹ and kept at −20° C. until analysis.

Internal standards were added to the samples. The samples were subjected to nano-UPLC-MS/MS, combined with RPM, as set out in Example 1.

The N-terminal and Truncation 2 peptide were detected from the EV-containing samples.

### Example 3

This example aimed to identify suitable internal standards for analysing disease-specific TDP-43 species in samples using mass spectrometry.

Internal standards for detecting the signature peptides of the following proteins were selected: TDP-43, neurofilament light peptide (NFL), Chitotriosidase-1 (CHIT1) and neurofilament heavy peptide (NFH). The signature peptides were selected based on its specificity, including the correct identification in chymotrypsin digested samples, small chromatographic shifts and zero divergence from the corresponding heavy-labelled peptide, which made them the strongest candidates for quantification.

### TDP-43

Internal standards for detecting the signature peptides of TDP-43 as set forth in Table 3 (SEQ ID NOs: 1-3, 9 and 52) were selected for use in this example

TABLE 3
Signature peptides of TDP-43

| SEQused | Brief | | Signal |
|---|---|---|---|
| ID NO | description | Sequence | seen |
| 1 | P_{N} | IRVTEDENDEPIE | 2+ |
| | | IPSEDDGTVL | |
| 2 | P_{C} | SGSNSGAAIGW | 1+ |
| 9 | P_{T1} | KLPNSKQSQDEPL | 2+ > 3+ |
| 3 | P_{T2} | SNRQLERSGRF | 3+ |
| 52 | P_{T3} | IIKGISVHISNAE | 4+ > 3+ > 2+ |
| | | PKHN | |

### NFL

The signature peptides of human neurofilament light peptide (NFL) provided in Table 4 (SEQ ID NOs: 22-28) were considered. It was found that the sequences as set forth in SEQ ID NOs 22, 23 and 24 provided the most useful signature peptides for NFL. Therefore, internal standards for detecting these signature peptide sequences were selected for use in this example.

TABLE 4
Signature peptides of NFL

| SEQ | Amino acid | | Expected |
|---|---|---|---|
| ID NO | positions* | Sequence | signal |
| 25 | 44 to 51 | SAPVSSSL | 1+ |
| 26 | 74 to 83 | SQVAAISNDL | 1+ |
| 27 | 84 to 94 | KSIRTQEKAQL | 4+, 3+ |
| 28 | 12 to 135 | RQKHSEPSRF | 5+, 4+, 3+ |
| 22 | 149 to 160 | AAEDATNEKQAL | 2+ |
| 23 | 184 to 191 | SREDAEGRL | 3+ |
| 24 | isoform unique | TSVGSITSGY | 1+ |

*Amino acid position numbering according to the human NFL sequence set forth in UniProtKB: P07196.

### CHIT1

The signature peptides of human Chitotriosidase-1 (CHIT1) provided in Table 5 (SEQ ID NOs: 29-34) were considered. It was found that the sequences as set forth in SEQ ID NOs: 29, 30 and 31 provided the most useful signature peptides for CHIT1. Therefore, internal standards for detecting these signature peptide sequences were selected for use in this example.

TABLE 5
Signature peptides of CHIT1

| SEQ | Amino acid | | Expected |
|---|---|---|---|
| ID NO | positions* | Sequence | signal |
| 29 | 140 to 155 | EYPGSQGSPAVDKERF | 3+ |
| 30 | [96 to 204 | EVDKIAQNL | 2+ |
| 31 | 274 to 294 | ASSSDTRVGAPATGSGTPGPF | 2+ |
| 32 | 310 to 324 | KGATKQRIQDQKVPY | 4+, 3+ |
| 33 | 390 to 397 | LPSGTPEL | 1+ |
| 34 | 398 to 419 | EVPKPGQPSEPEHGPSPGQDTF | 3+, 2+ |

*Amino acid position numbering according to the human CHIT1 sequence set forth in UniProtKB: Q13231.

### NFH

The signature peptides of human neurofilament heavy peptide (NFH) provided in Table 5 (SEQ ID NOs: 35-41) were considered. It was found that the sequences as set forth in SEQ ID NOs: 35, 36 and 37 provided the most useful signature peptides for NFH. Therefore, internal standards for detecting these signature peptide sequences were selected for use in this example.

TABLE 6
Signature peptides of NFH

| SEQ | Amino acid | | Expected |
|---|---|---|---|
| ID NO | positions* | Sequence | signal |
| 38 | 29 to 47 | ARKGGAGGTRSAAGSSSGF | 4+, 3+ |
| 35 | 51 to 65 | TRTSVSSVSASPSRF | 3+ |
| 36 | 66 to 77 | RGAGAASSTDSL | 2+ |
| 39 | 186 to 202 | DDEARQREEAEAAARAL | 4+ |
| 37 | 206 to 216 | AQEAEAARVDL | 2+ |
| 40 | 279 to 287 | EGHAVQSTL | 2+, 1+ |
| 41 | 350 to 360 | EDRHQADIASY | 3+, 2+ |

*Amino acid position numbering according to the human NFH sequence set forth in UniProtKB: P12036.

### Retention Time Standards

It was found that all the sequences as set forth in Table 7 (SEQ ID NOs: 42-51) provided useful retention time standards, and so they were selected for use in this example.

TABLE 7
Retention time standards

| SEQ | | Expected |
|---|---|---|
| ID NO | Sequence | iRT |
| 42 | KADGTVPESSGAGL | 0.00 |
| 43 | KSWPESDATVDAF | 54.62 |
| 44 | RVAYPSAADIGDY | 42.26 |
| 45 | RVIAAPDIITGF | 87.23 |
| 46 | RTVQENGGL | -24.92 |
| 47 | RIVGGPDIITGF | 70.52 |
| 48 | RYPEGGSIVTPY | 28.71 |
| 49 | RYPEAGTIVPTY | 33.38 |
| 50 | KANSEDVGTEAVF | 12.39 |
| 51 | KNSEVGAIIY | 19.79 |

An internal standard precursor containing the internal standards and retention time standards selected above was generated by QconCAT strategy (see 32). In brief, the QconCAT strategy involved culturing cells that express the internal standard precursor, where all possible sources of nitrogen for the cell culture in which the protein is synthesised are nearly 100% Nitrogen-15. This way, all the protein synthesised, including the internal standard precursor, were labelled with ¹⁵N. The resulting internal standard precursor has the sequence as set forth in SEQ ID NO: 21 (also referred to as the TDP43 QconCat Construct). An alternative way is to produce an unlabelled QConCAT and label it in vitro.

Cortex and spinal cord tissues samples were analysed using nano-UPLC-MS/MS combined with parallel reaction monitoring (PRM), as set out in Example 1. The QconCat Construct was digested with chymotrypsin, together with the samples, prior to mass spectrometry, and the individual peptides (N-labelled) were released and served in the same way as the internal standards in the samples to detect the endogenous light peptides.

### Example 4

It was investigated whether the disease-specific TDP-43 species can be identified in cerebrospinal fluid (CSF) and cortex samples using mass spectrometry.

The following samples were used:
Samples 1 to 15: cortex samples;
Sample 16: unprocessed, native CSF sample;
Sample 17: serum native sample
Samples 18 and 20: processed CSF samples to isolate extracellular vesicles (EV) (EV-containing samples);
Sample 19: albumin-depleted serum sample; and
Sample 21: pelleted CSF sample;
Sample 22: biofluids pooled.

The samples were prepared as described in Examples 1 and 2. The samples were subjected to nano-UPLC-MS/MS, combined with RPM, as set out in Example 1.

The results are set out in Table 8. The values in Table 8 represent the amount of the light peptide relative to the heavy peptide. The ratios were generated using a less conservative filter than used in Example 1. In this Example, the analysis required only 2 shared chromatographic points (non-zero in both light and heavy) per method and reported a ratio if there is even only one valid light-heavy transition pair rather than requiring at least 2.

TABLE 8
Ratio of peptides identified in samples.

| | 1 | 2 | 3 | 4 | 5 | 6 | 7 | 8 | 9 | 10 | 11 | 12 |
|---|---|---|---|---|---|---|---|---|---|---|---|---|
| IRVTEDEND | 0.04 | 0.02 | 0.04 | 0.22 | 0.11 | 0.04 | 0.18 | 0.14 | 0.11 | 0.14 | 0.16 | 0.30 |
| EPIEIPSEDDGTVL | | | | | | | | | | | | |
| (SEQ ID NO: 1) | | | | | | | | | | | | |
| SGSNSG | 0.03 | 0.02 | 0.04 | 0.06 | 0.02 | 0.01 | 0.04 | 0.03 | 0.02 | 0.03 | 0.03 | 0.05 |
| AAIGW | | | | | | | | | | | | |
| (SEQ ID NO: 2) | | | | | | | | | | | | |
| KLPNSKQ | 0.0002 | NA | 0.0002 | 0.0001 | NA | NA | 0.0001 | 0.0001 | 0.0001 | 0.0001 | 0.0001 | 0.0003 |
| SQDEPL | | | | | | | | | | | | |
| (SEQ ID NO: 9) | | | | | | | | | | | | |
| SNRQLERSGRF | 0.02 | 0.01 | 0.01 | 0.01 | 0.01 | 0.01 | 0.01 | 0.01 | 0.01 | 0.01 | 0.01 | 0.01 |
| (SEQ ID NO: 3) | | | | | | | | | | | | |
| IIKGISVH | 0.01 | 0.08 | 0.0004 | 0.82 | 0.31 | 1.15 | 0.01 | 0.01 | 0.50 | 0.01 | 4.47 | 0.56 |
| ISNAEPKHN | | | | | | | | | | | | |
| (SEQ ID NO: 52) | | | | | | | | | | | | |

| | 13 | 14 | 15 | 16 | 17 | 18 | 19 | 20 | 21 | 22 |
|---|---|---|---|---|---|---|---|---|---|---|
| IRVTEDEND | 0.03 | 0.23 | 0.05 | NA | NA | NA | NA | NA | NA | NA |
| EPIEIPSEDDGTVL | | | | | | | | | | |
| (SEQ ID NO: 1) | | | | | | | | | | |
| SGSNSG | NA | 0.04 | 0.03 | NA | NA | NA | NA | NA | NA | NA |
| AAIGW | | | | | | | | | | |
| (SEQ ID NO: 2) | | | | | | | | | | |
| KLPNSKQ | NA | NA | NA | NA | 0.05 | NA | NA | NA | NA | NA |
| SQDEPL | | | | | | | | | | |
| (SEQ ID NO: 9) | | | | | | | | | | |
| SNRQLERSGRF | 0.0003 | 0.01 | 0.01 | 0.01 | 0.01 | 0.0004 | 0.0001 | NA | NA | 0.01 |
| (SEQ ID NO: 3) | | | | | | | | | | |
| IIKGISVH | 0.99 | 0.0004 | 0.05 | NA | 0.15 | 0.04 | 0.09 | NA | NA | NA |
| ISNAEPKHN | | | | | | | | | | |
| (SEQ ID NO: 52) | | | | | | | | | | |

NA means no signal or light peptide is detected.

It can be seen that, in all cortex samples, the N-terminal (SEQ ID NO: 1), C-Terminal (SEQ ID NO: 2), Truncation 1 (SEQ ID NO: 9), Truncation 2 (SEQ ID NO: 3) and Truncation 3 (SEQ ID NO: 52) peptides were all detected. In serum native samples, Truncation 1 peptide (SEQ ID NO: 9), Truncation 2 peptide (SEQ ID NO: 3) and Truncation 3 peptide (SEQ ID NO: 52) were identified. In native CSF sample, Truncation 2 peptide (SEQ ID NO: 3) was identified. In EV-containing samples and albumin-depleted serum sample, Truncation 2 peptide (SEQ ID NO: 3) and Truncation 3 peptide (SEQ ID NO: 52) were identified. In summary, the data demonstrate that the signature peptides disclosed herein can be detected in various sample types, such as tissues samples (e.g. cortex) and biofluid samples (e.g. CSF, extracellular vesicles isolated from CSF, and serum). This therefore demonstrates the utility of the signature peptides disclosed herein in detecting aberrant TDP-43 species produced under pathological conditions which can be used to diagnose, monitor or provide prognosis of a condition characterised by TDP-43 proteinopathy.

### REFERENCES

1 Neumann M, et al. Science 314, 130-133 (2006).
2 Tan et al. Acta Neuropathol 133, 177-196 (2017).
3 Nelson P T, et al. Brain 142(6):1503-1527 (2019).
4 Hasegawa M, et al. Ann Neurol 64, 60-70 (2008).
5 Neumann M, et al. Acta Neuropathol 117, 137-149 (2009).
6 Laferriere F, et al. Nat Neurosci 22, 65-77 (2019).
7 Igaz L M, et al. Am J Pathol 173, 182-194 (2008).
8 Steinacker P, et al. Arch Neurol 65, 1481-1487 (2008).
9 Feneberg E et al. Mol Neurobiol 55, 7789-7801 (2018).
10 Kuiperij H B, et al. J Alzheimers Dis 55, 585-595 (2017).
11 Kasai T, et al. Ann Clin Transl Neurol 6, 2489-2502 (2019).
12 Feneberg E, et al. Amyotroph Lateral Scler Frontotemporal Degener 15, 351-356 (2014).
13 Kwong L K, et al. Acta Neuropathol Commun 2, 33 (2014).
14 Chiang et al. Sci Rep 6, 21581 (2016).
15 Thompson et al., Nature Reviews Neurology volume 12, pages 346-357(2016).
16 Kiernan et al., Lancet. 377(9769):942-55 (2011).
17 Rascovsky et al., Alzheimer Dis Assoc Disord. October-December; 21(4): S14-8 (2007).
18 Chan and White, Fmoc Solid Phase Peptide Synthesis—A Practical Approach. Oxford University Press (1999).
19 Sambrook et al. Molecular Cloning: a laboratory manual, 3rd edition, Cold Spring Harbour Laboratory Press (2001).
20 Current Protocols in Molecular Biology, Chapter 2, Ausubel et al., Eds., Greene Publishing and Wiley-Interscience, New York (1995).
21 Altschul J Mol Evol. 36(3):290-300 (1993).
22 Altschul et al Proc Natl Acad Sci USA. July; 87(14):5509-13 (1990).
23 Turner J A, et al. Front Neuroinform 4 (2010).
24 Kametani F, et al. Sci Rep 6, 23281 (2016).
25 Zhang Y J et al. Proc Natl Acad Sci USA 106, 7607-7612 (2009).
26 Yamashita T et al. Nat Commun 3, 1307 (2012).
27 Aizawa H et al. Acta Neuropathol 120, 75-84 (2010).
28 Wang X et al. Neurosci Lett 469, 112-116 (2010).
29 Sasaki S. J Neuropathol Exp Neurol 70, 349-359 (2011).
30 Braak H et al. Acta Neuropathol 133, 79-90 (2017).
31 Berning B A et al. Front Neurosci 13, 335 (2019).
32 Wohlgemuth et al., Proteomics March; 15(5-6): 862-879 (2015).

TABLE 2
Description of peptide origin and properties for PRM Validation

| | | | TDP-43 position | | Measured | | | | |
|---|---|---|---|---|---|---|---|---|---|
| | | | and sequence | | retention | | | | |
| | | | of heavy | | window | Ratio | | | |
| | | | isotope- | | (expected | (L/H) | SD | KL | Measured |
| Origin of | | Digestion | labelled | Labelled | retention | (mean ± | (mean ± | (mean ± | peptid- |
| Target | Enzyme | pattern | peptides* | Residue | time) | SD) | SD) | SD) | oforms |
| N-Terminal | Trypsin | Specific | 103 | +8 Da | 48 m-88 m | 24.7 ± | 0.88 ± | 0.02 ± | 2+ |
| discovery | | | TSDLIVLGLPW(K)a | | (end of | 43 | 0.64 | 0.02 | charge |
| cohort | | | (SEQ ID NO: 12) | | gradient) | | | | state |
| | | | | | (54 m) | | | | |
| Kametani | Trypsin | Specific | 276 | +10 Da | 30 m-48 m | 0.15 ± | 0.62 ± | 0.31 ± | 2+ |
| | | | FGGNPGGFGNQGGFG | | (35 m) | 0.16 | 0.19 | 0.3 | charge |
| | | | NS(R) | | | | | | state |
| | | | (SEQ ID NO: 14) | | | | | | |
| Kametani | Trypsin | C- | 276 | +3 Da | 37 m-88 m | 0.07 ± | 1.99 ± | 0.97 ± | 1+ & 2+ |
| | | Terminal | FGGNP(G)GFGNQGG | | (end of | 0.16 | 1.0 | 0.6 | charge |
| | | Non- | FGN | | gradient) | | | | states |
| | | specific | (SEQ ID NO: 16) | | (42 m) | | | | |
| Kametani | Trypsin | N- | 280 | +10 Da | 25 m-37 m | 0.05 ± | 1.05 ± | 0.65 ± | 2+ |
| | | Terminal | PGGFGNQGGFGN | | (30 m) | 0.09 | 1.6 | 0.6 | charge |
| | | Non- | S(R) | | | | | | state |
| | | specific | (SEQ ID NO: 18) | | | | | | |
| C-Terminal | Trypsin | Specific | 409 SS(G)WGM | +3 Da | 33 m-47 m | 0.46 ± | 0.73 ± | 0.01 ± | 1+ |
| In silico | | | (SEQ ID NO: 20) | | (38.5 m) | 0.37 | 0.60 | 0.02 | charge |
| prediction | | | | | | | | | state |
| | | | | | | | | | Oxidised |
| | | | | | | | | | M, |
| | | | | | | | | | 1+ |
| | | | | | | | | | charge |
| | | | | | | | | | state |
| N-Terminal | Chym | Specific | 5 | +10 Da | 43 m-88 m | 0.54 ± | 0.64 ± | 0.008 ± | 2+ |
| discovery | | | I(R)VTEDENDEPIE | | (end of | 0.62 | 0.2 | 0.01 | charge |
| cohort (P_{N}) | | | IPSEDDGTVL | | gradient) | | | | state |
| | | | (SEQ ID NO: 4) | | (47.5 m) | | | | |
| C-Terminal | Chym | Specific | 375 SGSNS(G)AAI | +3 Da | 30 m-45 m | 0.43 ± | 0.72 ± | 0.007 ± | 1+ |
| discovery | | | GW | | (40.5 m) | 0.3 | 0.3 | 0.006 | charge |
| cohort (P_{C}) | | | (SEQ ID NO: 5) | | | | | | state |
| Truncation | Chym | N- | 266 SNRQLERSG | +8 Da | 20 m-30 m | 0.017 ± | 0.45 ± | 0.014 ± | 3+ |
| 2 | | Terminal | (R)F | | (22.5 m) | 0.016 | 0.5 | 0.02 | charge |
| discovery | | Non- | (SEQ ID NO: 6) | | | | | | state |
| cohort (P_{T2}) | | specific | | | | | | | |
| Truncation | Chym | N- | 175 | +8 Da | 23 m-33 m | 0.03 ± | 0.54 ± | 0.71 ± | 2+ & 3+ |
| 1 (P_{T1}) | | Terminal | (K)LPNSKQSQDEPL | | (25.5 m) | 0.09 | 0.5 | 0.95 | charge |
| Kametani | | Non- | (SEQ ID NO: 10) | | | | | | states |
| | | specific | | | | | | | |

*The sequence of the peptides is given with the position of the first amino acid within the full-length TDP-43 amino acid sequence
() indicate heavy labelled amino acids (K Lysine, R Arginine, G Glycine)
Abbreviations: SD, Chromatographic point shift; KL, Kullback-Leibler Divergence; Chym, Chymotrypsin; given are mean and standard deviation.

Sequence listing

| SEQ ID | Brief description and sequence |
|---|---|
| 1 | A signature peptide of TDP-43 (P_{N}) |
| | IRVTEDENDEPIEIPSEDDGTVL |
| 2 | A signature peptide of TDP-43 (P_{C}) |
| | SGSNSGAAIGW |
| 3 | A signature peptide of TDP-43 (P_{T2}) |
| | SNRQLERSGRF |
| 4 | isotope-labelled amino acid sequence of SEQ ID NO: 1 |
| | I(R)VTEDENDEPIEIPSEDDGTVL |
| 5 | isotope-labelled amino acid sequence of SEQ ID NO: 2 |
| | SGSNS(G)AAIGW |
| 6 | isotope-labelled amino acid sequence of SEQ ID NO: 3 |
| | SNRQLERSG(R)F |
| 7 | C-terminal peptide of a truncated TDP-43 |
| | SNRQLERSGRFGGNPGGFGNQGGFGNSRGGGAGLGNNQGSNM |
| | GGGMNFGAFSINPAMMAAAQAALQSSWGMMGMLASQQNQS |
| | GPSGNNQNQGNMQREPNQAFGSGNNSYSGSNSGAAIGWGSAS |
| | NAGSGSGFNGGFGSSMDSKSSGWGM |
| 8 | full length TDP-43 |
| | MSEYIRVTEDENDEPIEIPSEDDGTVLLSTVTAQFPGACGLRYR |
| | NPVSQCMRGVRLVEGILHAPDAGWGNLVYVVNYPKDNKRKM |
| | DETDASSAVKVKRAVQKTSDLIVLGLPWKTTEQDLKEYFSTFG |
| | EVLMVQVKKDLKTGHSKGFGFVRFTEYETQVKVMSQRHMIDG |
| | RWCDCKLPNSKQSQDEPLRSRKVFVGRCTEDMTEDELREFFSQ |
| | YGDVMDVFIPKPFRAFAFVTFADDQIAQSLCGEDLIIKGISVHIS |
| | NAEPKHNSNRQLERSGRFGGNPGGFGNQGGFGNSRGGGAGLG |
| | NNQGSNMGGGMNFGAFSINPAMMAAAQAALQSSWGMMGML |
| | ASQQNQSGPSGNNQNQGNMQREPNQAFGSGNNSYSGSNSGAA |
| | IGWGSASNAGSGSGFNGGFGSSMDSKSSGWGM |
| 9 | A signature peptide of TDP-43 (P_{T1}) |
| | KLPNSKQSQDEPL |
| 10 | isotope-labelled amino acid sequence of SEQ ID NO: 9 |
| | (K)LPNSKQSQDEPL |
| 11 | A signature peptide of TDP-43 |
| | TSDLIVLGLPWK |
| 12 | isotope-labelled amino acid sequence of SEQ ID NO: 11 |
| | TSDLIVLGLPW(K) |
| 13 | A signature peptide of TDP-43 |
| | FGGNPGGFGNQGGFGNSR |
| 14 | isotope-labelled amino acid sequence of SEQ ID NO: 13 |
| | FGGNPGGFGNQGGFGNS(R) |
| 15 | A signature peptide of TDP-43 |
| | FGGNPGGFGNQGGFGN |
| 16 | isotope-labelled amino acid sequence of SEQ ID NO: 15 |
| | FGGNP(G)GFGNQGGFGN |
| 17 | A signature peptide of TDP-43 |
| | PGGFGNQGGFGNSR |
| 18 | isotope-labelled amino acid sequence of SEQ ID NO: 17 |
| | PGGFGNQGGFGNS(R) |
| 19 | A signature peptide of TDP-43 |
| | SSGWGM |
| 20 | isotope-labelled amino acid sequence of SEQ ID NO: 19 |
| | SS(G)WGM |
| 21 | An internal standard precursor: TDP43 QconCat Construct |
| | HHHHHHLKSWPESDATVDAFKADGTVPESSGAGLRVAYPSAA |
| | DIGDYRVIAAPDIITGFRTVQENGGLRIVGGPDIITGFRYPEGGSI |
| | VTPYRYPEAGTIVPTYKANSEDVGTEAVFKNSEVGAIIYAAEDA |
| | TNEKQALSREDAEGRLTSVGSITSGYEYPGSQGSPAVDKERFEV |
| | DKIAQNLASSSDTRVGAPATGSGTPGPFTRTSVSSVSASPSRFRG |
| | AGAASSTDSLAQEAEAARVDLIRVTEDENDEPIEIPSEDDGTVLS |
| | GSNSGAAIGWKLPNSKQSQDEPLSNRQLERSGRFIIKGISVHISN |
| | AEPKHN |
| 22 | A signature peptide of human NFL |
| | AAEDATNEKQAL |
| 23 | A signature peptide of human NFL |
| | SREDAEGRL |
| 24 | A signature peptide of human NFL |
| | TSVGSITSGY |
| 25 | A signature peptide of human NFL |
| | SAPVSSSL |
| 26 | A signature peptide of human NFL |
| | SQVAAISNDL |
| 27 | A signature peptide of human NFL |
| | KSIRTQEKAQL |
| 28 | A signature peptide of human NFL |
| | RQKHSEPSRF |
| 29 | A signature peptide of human CHIT1 |
| | EYPGSQGSPAVDKERF |
| 30 | A signature peptide of human CHIT1 |
| | EVDKIAQNL |
| 31 | A signature peptide of human CHIT1 |
| | ASSSDTRVGAPATGSGTPGPF |
| 32 | A signature peptide of human CHIT1 |
| | KGATKQRIQDQKVPY |
| 33 | A signature peptide of human CHIT1 |
| | LPSGTPEL |
| 34 | A signature peptide of human CHIT1 |
| | EVPKPGQPSEPEHGPSPGQDTF |
| 35 | A signature peptide of human NFH |
| | TRTSVSSVSASPSRF |
| 36 | A signature peptide of human NFH |
| | RGAGAASSTDSL |
| 37 | A signature peptide of human NFH |
| | AQEAEAARVDL |
| 38 | A signature peptide of human NFH |
| | ARKGGAGGTRSAAGSSSGF |
| 39 | A signature peptide of human NFH |
| | DDEARQREEAEAAARAL |
| 40 | A signature peptide of human NFH |
| | EGHAVQSTL |
| 41 | A signature peptide of human NFH |
| | EDRHQADIASY |
| 42 | A retention time standard for mass spectrometry |
| | KSWPESDATVDAF |
| 43 | A retention time standard for mass spectrometry |
| | KADGTVPESSGAGL |
| 44 | A retention time standard for mass spectrometry |
| | RVAYPSAADIGDY |
| 45 | A retention time standard for mass spectrometry |
| | RVIAAPDIITGF |
| 46 | A retention time standard for mass spectrometry |
| | RTVQENGGL |
| 47 | A retention time standard for mass spectrometry |
| | RIVGGPDIITGF |
| 48 | A retention time standard for mass spectrometry |
| | RYPEGGSIVTPY |
| 49 | A retention time standard for mass spectrometry |
| | RYPEAGTIVPTY |
| 50 | A retention time standard for mass spectrometry |
| | KANSEDVGTEAV |
| 51 | A retention time standard for mass spectrometry |
| | FKNSEVGAIIY |
| 52 | A signature peptide of TDP-43 (P_{T3}) |
| | IIKGISVHISNAEPKHN |

() indicates heavy labelled amino acids (K Lysine, R Arginine, G Glycine)

1. A method for analysing a sample from a subject, comprising detecting a signature peptide of a TDP-43 species in the sample, wherein the signature peptide is a fragment of TDP-43 that is 6 to 25 amino acids in length comprising an amino acid sequence having at least 90% sequence identity to the amino acid sequence of any of SEQ ID NOs: 1-3 and 52, and wherein the level of the signature peptide provides a diagnostic indicator of a subject having a condition characterised by TDP-43 proteinopathy.

2. The method of claim 1, wherein the signature peptide comprises or consists of the amino acid sequence of any of SEQ ID NOs: 1-3 and 52.

3. The method of claim 1 or claim 2:
(a) comprising detecting the signature peptide comprising the amino acid sequence of SEQ ID NO: 1 and the signature peptide comprising the amino acid sequence of SEQ ID NO:2; and optionally wherein:
the ratio of the level of the signal peptide comprising the amino acid sequence of SEQ ID NO: 2 to the level of the signal peptide comprising the amino acid sequence of SEQ ID NO: 1 in the sample is determined, wherein the ratio provides a diagnostic indicator of a subject having a condition characterised by TDP-43 proteinopathy;
(b) comprising detecting the signature peptide comprising the amino acid sequences of SEQ ID NO: 1 and the signature peptide comprising the amino acid sequences of SEQ ID NO: 3; and optionally wherein:
the ratio of the level of the signal peptide comprising the amino acid sequence of SEQ ID NO: 3 to the level of the signal peptide comprising the amino acid sequence of SEQ ID NO: 1 in the sample is determined, wherein the ratio provides a diagnostic indicator of a subject having a condition characterised by TDP-43 proteinopathy; and/or
(c) further comprises detecting a signature peptide of TDP-43 which is a fragment of TDP-43 that is 6 to 25 amino acids in length comprising SEQ ID NO: 9, and optionally wherein:
the ratio of the level of the signal peptide comprising SEQ ID NO: 9 to the level of the signal peptide comprising SEQ ID NO: 1 in the sample is determined, wherein the ratio provides a diagnostic indicator of a subject having a condition characterised by TDP-43 proteinopathy.

4. The method of any preceding claim, wherein the signature peptide is detected by mass spectrometry.

5. The method of claim 4, wherein the mass spectrometry is tandem mass spectrometry.

6. The method of claim 4 or claim 5, wherein the mass spectrometry is combined with separation of the peptides by liquid chromatography, such as ultra-high performance liquid chromatography (UHPLC).

7. The method of any preceding claim, further comprises:
(a) treating the sample with a protease, such as chymotrypsin, prior to detection of the signature peptide; and/or
(b) adding an internal standard to the sample, optionally wherein the internal standard is an isotope-labelled signal peptide.

8. The method of any one of claims 1 to 3, wherein the signature peptide is detected by an antibody capable of binding specifically to the signal peptide.

9. The method of any preceding claim, wherein the sample is:
(a) a tissue, such as cortex or spinal cord, or
(b) a fluid, such as a blood or a cerebrospinal fluid, optionally wherein the signature peptides are determined from extracellular vesicles isolated from the fluid sample.

10. The method of any of any preceding claim, further comprising determination of at least one of:
(a) a known biomarker for a condition characterised by TDP-43 proteinopathy, such as neurofilament light polypeptide, neurofilament heavy polypeptide and/or chitotriosidase-1, which is optionally detected by its one or more signature peptides;
(b) a known biomarker for a condition characterised by a proteinopathy other than TDP-43, such as tau;
(c) other information about the subject; and/or
(d) other diagnostic tests or clinical indicators for a condition characterised by TDP-43 proteinopathy.

11. A method for diagnosing if a subject has a condition characterised by TDP-43 proteinopathy, comprising analysing a sample from the subject according to the method of any of claims 1 to 10.

12. A method of discriminating a condition characterised by TDP-43 proteinopathy from a condition characterised by non-TDP-43 proteinopathy, comprising analysing a sample from the subject according to the method of any of claims 1 to 10.

13. The method of claim 11 or claim 12, wherein the condition characterised by TDP-43 proteinopathy is amyotrophic lateral sclerosis, frontotemporal dementia or Limbic-predominant age-related TDP-43 encephalopathy; and/or the condition characterised by non-TDP-43 proteinopathy is Alzheimer's disease or Parkinson's disease.

14. A method of discriminating amyotrophic lateral sclerosis from other forms of TDP-43 proteinopathy, e.g. Limbic-predominant age-related TDP-43 encephalopathy, comprising analysing a sample from the subject according to the method of any of claims 1 to 9.

15. A method of monitoring the efficacy of a therapy for a condition characterised by TDP-43 proteinopathy being administered to a subject, comprising analysing a sample from the subject according to the method of any of claims 1 to 10, wherein the level of the signal peptide is determined at two or more different points in time, with changing levels of the signal peptide over time indicating whether the disease is getting better or worse.

16. A method of determining the prognosis of a condition characterised by TDP-43 proteinopathy in a subject, comprising analysing a sample from the subject according to the method of any of claims 1 to 10, wherein the level of the signature peptide is determined at two or more different points in time, with changing levels of the signature peptide over time indicating whether the disease is getting better or worse.

17. A method of identifying a subject susceptible to a therapy for Alzheimer's disease, e.g. immunisation studies against Tau or Amyloid, or antisense treatment targeted to tau, comprising diagnosing if a subject has a condition characterised by TDP-43 proteinopathy according to the method of any of claims 1 to 10, wherein the subject having a condition characterised by TDP-43 proteinopathy is not susceptible to the therapy.

18. A method of treating a condition characterised by TDP-43 proteinopathy, comprising administering a nucleic acid molecule targeted to a gene causing TDP-43 proteinopathy, a neuroprotective agent, an anti-inflammatory agent, an agent that regulates inflammation or immune system, an agent that protect cells from accumulation of misfolded protein or excitatory stimuli, or high-caloric treatment to the subject, wherein the method further comprises diagnosing if a subject has a condition characterised by TDP-43 proteinopathy according to the method of any of claims 11 to 14, monitoring the efficacy of the therapy according to the method of claim 15, and/or determining the prognosis of the disease according to the method of claim 16.

19. A nucleic acid molecule targeted to a gene causing TDP-43 proteinopathy, a neuroprotective agent, an anti-inflammatory agent, an agent that regulates inflammation or immune system, or an agent that protect cells from accumulation of misfolded protein or excitatory stimuli for use in a method of treating a condition characterised by TDP-43 proteinopathy, comprising administering said molecule or agent to the subject, wherein the method further comprises diagnosing if a subject has a condition characterised by TDP-43 proteinopathy according to the method of any of claims 11 to 14, monitoring the efficacy of the therapy according to the method of claim 15, and/or determining the prognosis of the disease according to the method of claim 16.

20. A signature peptide of TDP-43 which is a fragment of TDP-43 that is 6 to 25 amino acids in length comprising an amino acid sequence having at least 90% sequence identity to the amino acid sequence of any of SEQ ID NOs: 1-3 and 52.

21. The signature peptide of claim 20, comprising or consisting of the amino acid sequence of any of SEQ ID NOs: 1-3 and 52.

22. An internal standard for detecting a signature peptide of TDP-43, wherein the internal standard is an isotope-labelled signal peptide of claim 20 or 21, such as any one of SEQ ID NOs: 4-6.

23. An internal standard precursor, comprising the internal standard of claim 22 and an internal standard for detecting a signature peptide of a known marker of TDP43 proteinopathy, such as neurofilament light polypeptide (NFL), neurofilament heavy polypeptide (NFH) and/or chitotriosidase-1 (CHIT1).

24. The internal standard precursor of claim 23, consisting of an isotope-labelled amino acid sequence of SEQ ID NO: 21.

25. A fragment of TDP-43 which is 100 to 180 amino acids in length comprising an amino acid sequence having at least 90% sequence identity to the amino acid sequence of SEQ ID NO: 7.

26. A polynucleotide encoding the peptide of any of claims 20, 21, 23 and 25.

27. An antibody capable of binding specifically to the peptide of any of claims 20, 21 and 25.

28. Use of the signature peptide of claim 20 or 21, the internal standard of claim 22, the internal standard precursor of claim 23 or 24, the TDP-43 fragment of claim 25, the polynucleotide of claim 26, or the antibody of claim 27, to diagnose or provide prognosis for a condition characterised by TDP-43 proteinopathy.
